(19) United States
(12) Patent Application Publication
(10) Pub. No.: US 2003/0229082 A1
(43) Pub. Date: Dec. 11, 2003

(54) Inhibition of RNA function

(75) Inventors: Thomas L. James, Nicasio, CA (US); Kenneth E. Lind, San Francisco, CA (US); Zhihua Du, Sunnyvale, CA (US); B. Matija Peterlin, San Francisco, CA (US); Koh Fujinaga, University Heights, OH (US); Rodney Kiplin Guy, Concord, CA (US); Peter Bawden Madrid, San Francisco, CA (US); Moriz Mayer, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA

(21) Appl. No.: 10/346,397

(22) Filed: Jan. 16, 2003

Related U.S. Application Data
(60) Provisional application No. 60/349,769, filed on Jan. 16, 2002.

(51) Int. Cl. A61K 31/542; A61K 31/538; A61K 31/498; A61K 31/4745; A61K 31/4015; A61K 31/343

(52) U.S. Cl. 514/224.8; 514/229.8; 514/250; 514/283; 514/424; 514/468

(57) ABSTRACT

Inhibition of RNA function, and treatment or control of diseases or conditions, e.g. infectious diseases such as viruses and viral infections (including HIV) and microbial infections, by the contacting of the RNA with a compound having a central or core structure comprising three fused rings containing from 12 to 15 ring atoms, the central ring including at least one heteroatom selected from nitrogen, oxygen and sulfur, the atoms of the three-ring core structure being optionally substituted with substituents such as halogens, cyano, and/or various substituted or unsubstituted aliphatic and/or heteroaliphatic moieties, or contacting the RNA with yohimbine, usnic acid or N-{4-[2,5-Dioxo-1-(4-trifluoromethoxy-phenyl)-pyrrolidin-3-yl]-phenyl}-2,2,2-trifluoro-acetamide. Preferred compounds are various phenothiazines, including both known and novel compounds.

### STATEMENT AS TO RIGHTS TO INVENTIONS MADE UNDER FEDERALLY SPONSORED RESEARCH AND DEVELOPMENT

This invention was made with government support under Grant No. A146967 of the National Institutes of Health. The government has certain rights in this invention.

### BACKGROUND AND PRIOR ART

This invention relates to inhibition of the functioning of RNA molecules (termed herein “inhibition of RNA function”) by compounds of a type not previously known to have such an effect. The invention further relates to inhibition of function of microbial RNA and/or of viral RNA, including retroviral RNA, and to prevention or inhibition of the replication of viruses including HIV by such compounds, in the last-mentioned case through targeting and binding of compounds in question to the HIV RNA genome, particularly at the TAR (transactivation response) element on the RNA genome of HIV.

The invention further relates to treatment or inhibition of a viral or microbial infection, disease or condition in a patient or subject, more particularly in a mammal. However, the invention is not limited to treatment of subjects but encompasses the inhibition of RNA function per se and in various environments such as cells or cell cultures.

Ribonucleic acids are a promising, yet relatively untapped, target for drug design. Currently, most drugs on the market target proteins. Yet, in many ways RNA may be a better target than a protein, since it is upstream in the translation pathway. Inhibiting a single RNA molecule could prevent the production of thousands of proteins. As more three-dimensional (3D) RNA structures become available, unique binding sites will be defined for targeting.

There have been some attempts to discover drugs that interact with RNA. Such work is described, for instance, in Chen, et al., (1997), Structure-Based Discovery of Ligands Targeted to the RNA Double Helix, Biochemistry 36, 11402-11407; Hermann, et al. (1999), Docking of Cationic Antibiotics to Negatively Charged Pockets in RNA Folds, J. Med. Chem. 42, 1250-1261; and particularly Xavier, et al. (2000), RNA as a Drug Target: Methods for Biophysical Characterization and Screening, Trends in Biotechnology 18, 349-356. These authors focused primarily on antibacterial agents, since the target of some clinically important antibacterial drugs, originally discovered by soil sample screening, was found to be bacterial RNA. In general, the active agents found in that work, which are aminoglycosides, are valuable, but they have undesirable features. For instance, they interact with other sites on nucleic acids in human cells, and bacteria develop resistance. Consequently, it is desirable to identify new classes of compounds for drug development and RNA targets that cannot be altered due to function.

Thus, the determination that compounds inhibit the functioning of bacterial RNA has significance in attaining control of microbial infections, and serves to identify compounds that can function as antimicrobials. Likewise, the determination that a compound can inhibit the functioning of viral RNA provides identification of compounds that possess anti-viral activity. Compounds having anti-microbial or anti-viral activity may be used in prophylactic or preventive care and in treating microbial or viral infections existing in subjects.

Compounds that inhibit interactions of RNA with ligands by binding to the RNA and successfully competing with the natural protein or RNA ligand of the RNA may be important, e.g., in treating or preventing a disease or abnormal condition, such as an infection or unchecked growth. Identification of compounds that inhibit interactions of RNA with ligands or otherwise inhibit the functioning of RNA, thus can lead to identification of new compounds that may be used to treat or prevent such diseases or conditions, or of such new uses for known compounds.

Three principal types of RNA exist in cells; messenger RNA, transfer RNA and ribosomal RNA. The messenger RNAs (mRNA) each contain enough information from the parent DNA molecule to direct the synthesis of one more proteins. Each has attachment sites for tRNAs and rRNA. The transfer RNAs (tRNA) each recognize a specific codon of three nucleotides in a strand of mRNA, the amino acid specified by the codon, and an attachment site on a ribosome. Each tRNA is specific for a particular amino acid and functions as an adaptor molecule in protein synthesis, supplying that amino acid to be added to the distinctive polypeptide chain. Subunits of ribosomal RNA (rRNA) form components of ribosomes, the “factories” where protein is synthesized. The subunits have attachment sites for mRNA and the polypeptide chain. The rRNAs regulate aminoacyl-tRNA binding, mRNA binding, and the binding of the initiation, elongation, and termination factors; peptide bond formation; and translocation.

The RNAs share a common overall structure, though each kind of RNA has a unique detailed substructure. Generally, RNA is a linear, repetitive polymer in which nucleotide subunits are covalently linked to each other in sequence. Each nucleotide subunit consists of a base linked to the ribose-phosphate of the polymeric backbone. The bases in RNA are adenine(A), uracil (U), guanine (G), and cytosine (C). The sequence of bases imparts specific function to each RNA molecule. Nucleotide bases from different parts of the same or different RNA molecules recognize and noncovalently bond with each other to form base pairs. Since RNAs generally are a single covalent strand, base pairing interactions are usually intrastranded, in contrast to the interstrand base pairing of DNA. These noncovalent bonds play a major part in determining the three-dimensional structure of each of the RNAs and the interaction of RNA molecules with each other and with other molecules. The 2′ hydroxyl group also influences the chemical properties of RNA, imposing stereochemical constraints on the RNA structure, by restricting the ribose conformation in oligomeric RNA molecules to the C3′-endo conformation, in contrast to DNA, where the sugars freely interconvert between the C3′-endo and C2′-endo puckered conformations.

Since RNA is critical to protein synthesis and the transfer of genetic information encoded in the deoxyribonucleic acid (DNA) of eukaryotic cells, bacteria, and viruses, it represents a potential mechanism by which all pathogenic agents can be inhibited. To this time, however, little progress has been made in identifying a means by which RNA can be inhibited specifically.

Nucleic acids, and in particular RNAs, are capable of folding into complex tertiary structures that include bulges, loops, triple helices and pseudoknots, which can provide binding sites for in vivo ligands, such as proteins and other RNAs. RNA-protein and RNA-RNA interactions are important in a variety of cellular functions, including transcription, RNA splicing, RNA stability, gene regulation and translation. As used herein, “ligand” refers to a molecule, e.g., a protein or RNA molecule that binds to a defined binding site on the target RNA.

In recent years, several drugs designed using three-dimensional protein structures were approved for clinical use [Charifson, et al. (1997), Recent Successes and Continuing Limitations in Computer-Aided Drug Design; in Practical Application of Computer-Aided Drug Design Edited (Charifson, P. S., ed.), pp. 1-37, Marcel Dekker, Inc., New York].

There has been little reported research on drug discovery using unique three-dimensional RNA structures. Some recent work is reported in Filikov, et al., (2000), Identification of ligands for RNA targets via structure-based virtual screening: HIV-1 TAR, Journal of Computer-Aided Molecular Design 14, 593-610 and James, et al. (2000), Three-Dimensional RNA Structure-Based Drug Discovery [in Structure, Motion, Interaction and Expression of Biological Macromolecules (Sarma, R. H., ed.), pp. 201-205, Adenine Press, New York, N.Y.].

Acquired immunodeficiency syndrome (AIDS) is caused by the human immunodeficiency virus, type 1 (HIV-1). Unique structures on the HIV-1 RNA genome that play essential roles in viral replication are good targets for drug design. One such site is the transactivation response (TAR) element. It binds the viral transactivation protein, Tat, which regulates expression of all viral genes by increasing production of mature, full-length viral RNA. The HIV-1 genome encodes Tat. TAR RNA forms a short bulged stem-loop structure (see FIG. 1) at the 5′-end of all viral transcripts. Essential features of Tat and TAR have been delineated. In particular, the 5′ bulge and adjacent stem region are critical for Tat binding. Both TAR and Tat form a ternary complex with human cyclin T1 (CycT1), resulting in increased rates of elongation of transcription on the HIV-1 genome.

Interrupting the interaction between Tat and TAR has been found to block HIV-1 replication in infected cells. Hsu, et al., Inhibition Of HIV Replication In Acute and Chronic Infections in vitro by A Tat Antagonist, Science 254, 1799-1802 (1991) studied the effects of the compound Ro-3335 [7-chloro-5-(2-pyrryl)-3H-1,4,-benzodiazepin-2(H)-one] as an inhibitor of this interaction. Other work was carried out by Sullenger et al., Analysis of Trans-Acting Response Decoy RNA-Mediated Inhibition of Human Immunodeficiency Virus Type-1 Transactivation, J. Virol., 65, 6811-6816 (1991); Dayton, et al., Cell 44, 941-947 (1986); and Fisher et al., Nature 320, 367 (1986). Mei, et al., (1998) Inhibitors of Protein-RNA Complexation That Target the RNA: Specific Recognition of Human Immunodeficiency Virus Type 1 TAR RNA by Small Organic Molecules, Biochemistry 37, 14204-14212, described three small-molecule organic inhibitors of the HIV-1 Tat-TAR interaction. Others have determined that analogs of amino acids or nucleotides and aminoglycosides can inhibit the binding of Tat to TAR. Aminoglycoside-arginine conjugates have also been found by Litovchick, A., et al. (2000), Aminoglycoside-arginine conjugates that bind TAR RNA: Synthesis, Characterization, and Antiviral Activity, Biochemistry 39, 2838-2852, to be effective inhibitors of the Tat-TAR interaction. While the best of the other compounds bind with micromolar affinity, some aminoglycosides conjugated with multiple arginines achieved binding in the 20-400 nM range.

The TAR-Tat protein interaction is essential for HIV replication, because (as described in above-mentioned references) binding of Tat to TAR is required for activating transcription of the HIV genome. Finding agents that disrupt the Tat-TAR interaction therefore would provide a strategy to inhibit HIV replication. Finding agents that disrupt or inhibit replication of RNA of other types similarly can identify such agents that may be used to treat other diseases, including diseases of viral or microbial origin.

Similarly, binding to distinct sites on ribosomes can result in inhibition of microbial growth or infection. This is the general mode of action of many antimicrobials, especially antibacterials, such as the aminoglycosides (e.g., neomycin), chloramphenicol (amino acid derivative), erythromycin (macrolide), thiostrepton (thiopeptide) and tetracycline. Xavier et al., Trends in Biotechnology, supra. The ribosomal A-site is one such binding site. Inhibition of binding to this site (one aspect of inhibiting the functioning of the RNA) can result in effective inhibition or preventing of the spread of a microbial (e.g. bacterial, fungal, or protozoal) infection or undesirable condition, and compounds that are found to inhibit such binding can possess this effect.

### SUMMARY OF THE INVENTION

In its most general aspect, this invention comprises a method of inhibiting RNA function comprising contacting an RNA molecule, or RNA generally, with a pharmacologically effective inhibitory amount of a compound as described herein.

In another aspect, this invention comprises a method for inhibition of RNA function comprising contacting cells that comprise or contain RNA with a pharmacologically effective inhibitory amount of such a compound.

In another aspect, this invention comprises a method for inhibiting a microbial infection in a subject or a cell comprising administering to said subject or cell a pharmacologically effective inhibitory amount of such a compound.

In still another aspect, this invention comprises a method for inhibiting a viral infection in a subject or a cell comprising administering to said subject or cell a pharmacologically effective inhibitory amount of such a compound.

In yet another aspect, this invention comprises a method for inhibition of function of viral or microbial RNA comprising administering to a subject a pharmacologically effective inhibitory amount of such a compound.

Viruses whose replication may be inhibited by use of this invention include retroviruses, particularly HIV, as well as polio viruses, rhinoviruses (for example, responsible for the common cold), enteroviruses and hepatitis C. Microbial infections that may be inhibited include bacterial, fungal and protozoal infections.

The majority of compounds of this invention have a central or core structure comprising three fused rings containing from 12 to 15 ring atoms, the central ring including at least one heteroatom selected from nitrogen, oxygen and sulfur, the atoms of the three-ring core structure being optionally substituted with substituents such as halogens, cyano, and/or various substituted or unsubstituted aliphatic and/or heteroaliphatic moieties.

Most compounds of this invention have the general formula (I):

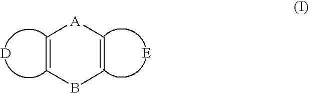

in which:

A is O; NR₁; S(O)ₙ where n is 0, 1 or 2; CR₂R₃; or —A′—CR₄R₅, where A′ is O, NR₁, S(O)ₙ wherein n is 0, 1 or 2 or CR₆R₇, wherein R₁ through R₇ are independently hydrogen or optionally substituted aliphatic or heteroaliphatic groups, preferably such groups having from 1-6 carbon atoms;

B is N—R, O, S or CR₈R₉ in which R is hydrogen or an optionally substituted aliphatic or heteroaliphatic group and R₈ and R₉ are independently hydrogen or optionally substituted aliphatic or heteroaliphatic groups, or when B is N—R, then R, the nitrogen atom of B, and two carbon atoms on the ring adjacent to the nitrogen atom, together comprise a ring having the formula —N—C═C—C(OH)═C(COCH₃)—C(O)—;

provided that at least one of A and B comprises an oxygen, sulfur or nitrogen ring atom;

D and E, together with the carbon atoms to which they are bonded, independently comprise rings of 5-7 atoms selected from C, N, S and O, optionally substituted, wherein each ring includes at least one double bond; and wherein substituents on atoms of groups D and E are as described herein;

and pharmaceutically acceptable salts thereof.

Also within the scope of this invention is such use of yohimbine, of usnic acid, and of the compound N-{4-[2,5-Dioxo-1-(4-trifluoromethoxy-phenyl)-pyrrolidin-3-yl]-phenyl}-2,2,2-trifluoro-acetamide (“Maybridge 15091”, available from Maybridge PLC, England) to inhibit RNA replication and/or as antimicrobial or antiviral agents.

Yet another aspect of the invention comprises a method of forming an RNA complex with a compound of the invention, the method comprising contacting an RNA molecule with a compound of the invention. Still another aspect of this invention comprises complexes of the above-identified compounds with the TAR region.

Some compounds within the above definitions are known compounds. Some of these known compounds, such as the phenothiazines promazine, acetopromazine, chlorpromazine, prochlorperazine and trifluoperazine, are known pharmaceuticals, used as anti-nausea agents and as antipsychotic drugs. Other compounds within the scope of this invention are known compounds as such, but have not heretofore been shown to possess pharmaceutical or pharmacological properties. Yet other compounds of this invention are novel compounds that have been found to inhibit RNA function and consequently inhibit viral and microbial infections. A still further aspect of this invention comprises pharmaceutical compositions that contain a pharmaceutically or pharmacologically effective amount of a compound that is either novel or is known but that has not hitherto been identified as having a pharmaceutical use.

### DESCRIPTION OF THE DRAWINGS

FIG. 1 depicts the TAR (transactivation response element) region of the RNA of the HIV genome.

FIG. 2 depicts bulge and/or loop regions of RNA of the ribosomal A-site, of the polio virus, of the dimer linkage site stem-loop 1 (DLS SL1) of the HIV-1 virus, of the coxsackievirus B3 (CVB3) virus, and of the TAR region of the HIV genome.

FIG. 3 depicts structures of some representative phenothiazine compounds of this invention.

FIG. 4 depicts the three-dimensional structure of the TAR-acetylpromazine complex of this invention, showing the relationship of an acetylpromazine molecule to TAR RNA.

### DETAILED DESCRIPTION OF THE INVENTION

According to this invention, it has been found that certain compounds have the capability of inhibiting the functioning of RNA and of inhibiting infectious diseases in general, and particularly viral and microbial infections. These compounds have not heretofore been known to possess such activity, though some may be known or may belong to well-known classes of compounds having other types of pharmacological activity and uses.

More particularly, compounds of this invention have been found to bind to a bulge and/or loop in RNA. Such binding, if by an effective amount of the compound, inhibits binding of another ligand, such as a protein, to the RNA that is necessary for the biological function of the RNA, and thus inhibits that function of the RNA. If the RNA is a viral or microbial RNA, the inhibition of function results in an inhibition of replication of the virus or of the microbial infection itself. As all natural RNAs of interest as drug targets have a bulge or loop involved in the biological function of the RNA, this invention thus includes the inhibition of the function of that RNA by contacting the RNA (which may be per se, in a cell or in a subject) with a pharmacologically effective inhibitory amount of a compound as described herein.

The compound may also bind to a region of the RNA that is adjacent to the bulge and/or the loop, or inhibit RNA function in some other manner.

More particularly, the present invention involves identifying compounds that bind to a target RNA at a ligand binding site and inhibit the interaction of that RNA with one or more in vivo ligands. The compounds of the invention thus are useful for inhibiting the formation of a specific RNA-ligand complex in vivo.

The term “inhibition of RNA function” (or “functioning”), as used herein, refers to a decrease in one or more functions of the RNA, such as transcription, regulation, translation, attachment of amino acids, activation of subsequent amino acids as required to form peptides, binding of initiation, elongation and termination factors, peptide bond formation, or translocation. The term “inhibition” as used herein and as applied to viral replication or microbial infection is meant to include partial and total inhibition of viral replication as well as decreases in the rate of the viral replication or microbial infection.

In some embodiments, compounds of the invention are useful for increasing or decreasing the translation of messenger RNAs (“mRNAs”), e.g., increasing or decreasing protein production, by binding to one or more regulatory elements in the 5′ untranslated region, the 3′ untranslated region, or the coding region of the mRNA. Compounds that bind to mRNA can, inter alia, increase or decrease the rate of mRNA processing, alter its transport through the cell, prevent or enhance binding of the mRNA to ribosomes, suppressor proteins or enhancer proteins, or alter mRNA stability. Accordingly, compounds that increase or decrease mRNA translation can be used to treat or prevent disease.

To put it another way, the methods of the invention can be used to identify mRNA-binding compounds for increasing or decreasing the production of a protein, thus treating or preventing a disease associated with decreasing or increasing the production of said protein, respectively. These include diseases in mammals, including cats, dogs, swine, horses, goats, sheep, cattle, primates and humans. For example, diseases associated with protein overproduction, such as amyloidosis, or with the production of mutant proteins, such as cystic fibrosis, can be treated or prevented by decreasing translation of the mRNA that codes for the overproduced protein, thus inhibiting production of the protein. Conversely, the symptoms of diseases associated with decreased protein function, such as hemophilia, may be treated by increasing translation of mRNA coding for the protein whose function is decreased, e.g., factor IX in some forms of hemophilia.

Compounds of the invention may bind to mRNAs coding for a variety of proteins with which the progression of diseases in mammals is associated. These mRNAs include, but are not limited to, those coding for amyloid protein and amyloid precursor protein; anti-angiogenic proteins such as angiostatin, endostatin, METH-1 and METH-2; clotting factors such as Factor IX, Factor VIII, and others in the clotting cascade; collagens; cyclins and cyclin inhibitors, such as cyclin dependent kinases, cyclin D1, cyclin E, WAF1, cdk4 inhibitor, and MTS1; cystic fibrosis transmembrane conductance regulator gene (CFTR); cytokines such as IL-1, IL-2, IL-3, IL-4, IL-5, IL-6, IL-7, IL-8, IL-9, IL-10, IL-11, IL-12, IL-13, IL-14, IL-15, IL-16, IL-17 and other interleukins; hematopoietic growth factors such as erythropoietin; colony stimulating factors such as G-CSF, GM-CSF, M-CSF, SCF and thrombopoietin; growth factors such as BNDF, BMP, GGRP, EGF, FGF, GDNF, GGF, HGF, IGF-1, IGF-2, KGF, myotrophin, NGF, OSM, PDGF, somatotrophin, TGF-.beta., TGF-.alpha. and VEGF; antiviral cytokines such as interferons, antiviral proteins induced by interferons, TNF-.alpha., and TNF-.beta.; enzymes such as cathepsin K, cytochrome p-450 and other cytochromes, farnesyl transferase, glutathione-s transferases, heparanase, HMG CoA synthetase, n-acetyltransferase, phenylalanine hydroxylase, phosphodiesterase, ras carboxyl-terminal protease, telomerase and TNF converting enzyme; glycoproteins such as cadherins, e.g., N-cadherin and E-cadherin; cell adhesion molecules; selectins; transmembrane glycoproteins such as CD40; heat shock proteins; hormones such as 5-.alpha. reductase, atrial natriuretic factor, calcitonin, corticotrophin releasing factor, diuretic hormones, glucagon, gonadotropin, gonadotropin releasing hormone, growth hormone, growth hormone releasing factor, somatotropin, insulin, leptin, luteinizing hormone, luteinizing hormone releasing hormone, parathyroid hormone, thyroid hormone, and thyroid stimulating hormone; proteins involved in immune responses, including antibodies, CTLA4, hemagglutinin, MHC proteins, VLA-4, and kallikrein-kininogen-kinin system; ligands such as CD4; oncogene products such as sis, hst, protein tyrosine kinase receptors, ras, abl, mos, myc, fos, jun, H-ras, ki-ras, c-fms, bcl-2, L-myc, c-myc, gip, gsp, and HER-2; receptors such as bombesin receptor, estrogen receptor, GABA receptors, growth factor receptors including EGFR, PDGFR, FGFR, and NGFR, GTP-binding regulatory proteins, interleukin receptors, ion channel receptors, leukotriene receptor antagonists, lipoprotein receptors, opioid pain receptors, substance P receptors, retinoic acid and retinoid receptors, steroid receptors, T-cell receptors, thyroid hormone receptors, TNF receptors; tissue plasminogen activator; transmembrane receptors; transmembrane transporting systems, such as calcium pump, proton pump, Na/Ca exchanger, MRP1, MRP2, P170, LRP, and cMOAT; transferrin; and tumor suppressor gene products such as APC, brca1, brca2, DCC, MCC, MTS1, NF1, NF2, nm23, p53 and Rb.

The compounds of the invention thus may be useful for treating or preventing a disease in mammals, including cats, dogs, swine, horses, goats, sheep, cattle, primates and humans. Such diseases include, but are not limited to, amyloidosis, hemophilia, Alzheimer's disease, atherosclerosis, cancer, giantism, dwarfism, hypothyroidism, hyperthyroidism, inflammation, cystic fibrosis, autoimmune disorders, diabetes, aging, obesity, neurodegenerative disorders, and Parkinson's disease. The compounds of this invention also are useful in treating or preventing various infectious diseases, including diseases caused by viral or microbial infections (e.g., bacterial, fungal and/or protozoal infections including (but not limited to) HIV infection, AIDS, human T-cell leukemia, SIV infection, FIV infection, feline leukemia, hepatitis A, hepatitis B, hepatitis C, Dengue fever, malaria, rotavirus infection, severe acute gastroenteritis, diarrhea, encephalitis, hemorrhagic fever, syphilis, legionella, whooping cough, gonorrhea, sepsis, influenza, pneumonia, tinea infection, candida infection, meningitis and the common cold.

In other embodiments, compounds of the invention are useful for preventing the interaction of an RNA, such as a transfer RNA (“tRNA”), an enzymatic RNA or a ribosomal RNA (“rRNA”), with a protein or with another RNA, thus preventing, e.g., assembly of an in vivo protein-RNA or RNA-RNA complex that is essential for the viability of a cell. The term “enzymatic RNA,” as used herein, refers to RNA molecules that are either self-splicing, or that form an enzyme by virtue of their association with one or more proteins, e.g., as in RNAse P, telomerase or small nuclear ribonucleoprotein particles. For example, inhibition of an interaction between rRNA and one or more ribosomal proteins may inhibit the assembly of ribosomes, rendering a cell incapable of synthesizing proteins. In addition, inhibition of the interaction of precursor rRNA with ribonucleases or ribonucleoprotein complexes (such as RNAse P) that process the precursor rRNA prevent maturation of the rRNA and its assembly into ribosomes. Similarly, a tRNA:tRNA synthetase complex may be inhibited by test compounds identified by the methods of the invention such that tRNA molecules do not become charged with amino acids. Such interactions include, but are not limited to, rRNA interactions with ribosomal proteins, tRNA interactions with tRNA synthetase, RNase P protein interactions with RNase P RNA, and telomerase protein interactions with telomerase RNA.

In other embodiments, compounds of the invention are useful for treating or preventing a viral, bacterial, protozoal or fungal infection. For example, as shown below, compounds of the invention may bind to a loop of the ribosomal A-site and thus have an inhibitory effect on replication of that RNA, and consequently on associated microbial infections. Examples of microbial target RNAs useful in the present invention for identifying antiviral, antibacterial, antiprotozoal and antifungal compounds include, but are not limited to, general antiviral and anti-inflammatory targets such as mRNAs of INF .alpha., INF .gamma., RNAse L, RNAse L inhibitor protein, PKR, tumor necrosis factor, interleukins 1-15, and IMP dehydrogenase; internal ribosome entry sites; HIV-1 CT rich domain and RNAse H mRNA; HCV internal ribosome entry site, which is required to direct translation of HCV mRNA; rotavirus NSP3 binding site, which binds the protein NSP3 that is required for rotavirus mRNA translation; HBV epsilon domain; Dengue virus 5′ and 3′ untranslated regions, including IRES, INF .alpha., INF .beta. and INF .gamma.; plasmodium falciparum mRNAs; the 16S ribosomal subunit ribosomal RNA and the RNA component of RNAse P of bacteria; and the RNA component of telomerase in fungi and cancer cells.

Most of the compounds of this invention are characterized by having a central or core structure comprising three fused rings containing from 12 to 15 atoms in the rings, the central ring including at least one heteroatom selected from nitrogen, oxygen and sulfur, the atoms of the core structure being optionally substituted with halogen, cyano, or optionally substituted aliphatic and/or heteroaliphatic moieties. The compounds of this invention include as well pharmaceutically acceptable salts of such compounds.

Most compounds of this invention have the general formula (I)

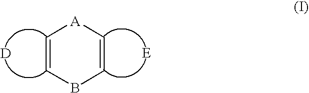

in which:

A is O; NR₁; S(O)ₙ where n is 0, 1 or 2; CR₂R₃; or —A′—CR₄R₅, where A′ is O, NR₁, S(O)ₙ wherein n is 0, 1 or 2 or CR₆R₇, wherein R₁ through R₇ are independently hydrogen or optionally substituted aliphatic or heteroaliphatic groups having from 1-6 carbon atoms;

B is N—R, O, S or CR₈R₉ in which R is hydrogen or an optionally substituted aliphatic or heteroaliphatic group and R₈ and R₉ are independently hydrogen or optionally substituted aliphatic or heteroaliphatic groups, or when B is N—R, then R, the nitrogen atom of B, and two carbon atoms on the ring adjacent to the nitrogen atom, together comprise a ring having the formula —N—C═C—C(OH)═C(COCH₃)—C(O)—;

provided that at least one of A and B comprises an oxygen, sulfur or nitrogen ring atom;

D and E, together with the carbon atoms to which they are bonded, independently comprise optionally substituted rings of 5-7 atoms selected from C, N, S and O, optionally substituted, wherein each ring includes at least one double bond; wherein substituents on atoms of groups D and E are as described herein;

and pharmaceutically acceptable salts thereof.

In a preferred class of compounds, D and E each comprise chains of four carbon atoms, optionally substituted and containing two double bonds. Groups D and E thus comprise, together with the carbon atoms to which they are bound, six-membered rings containing at least two double bonds (and preferably three double bonds). In one embodiment, D and E are unsubstituted. In another embodiment, one of D and E is mono-substituted. In a third embodiment, both D and E are mono-substituted.

This preferred class of compounds includes phenothiazines (A is sulfur, and B is N—R), which have the following general structure (II) in which the side rings are optionally substituted as above, and which constitutes a preferred subclass of compounds of this invention:

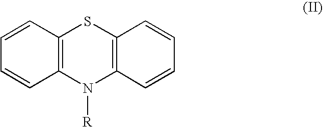

Also included in this preferred class of compounds are the thioxanthenes (A of Formula I is sulfur and B is CR₈R₉), thianthrenes (A and B are both sulfur), phenoxazines (A is oxygen, B is N—R), phenazines (A and B are both nitrogen), phenoxathiins (A is sulfur and B is oxygen) and the benzepines, which are characterized by having a 7-member nitrogen-containing central ring, and include such subclasses as dibenzodiazepines (A is N═CR₄ and B is N—R) and dibenzoxazepines (A is N═CR₄ and B is oxygen).

Atoms on the side rings of the phenothiazines and other compounds mentioned above are optionally substituted, as described below.

Preferred compounds of this invention include pharmaceutically acceptable salts of the above-described compounds.

Also included within the scope of this invention is the use of three other compounds that have been found to inhibit RNA function and act as antimicrobial and/or antiviral agents. These are:

yohimbine:

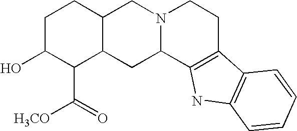

usnic acid

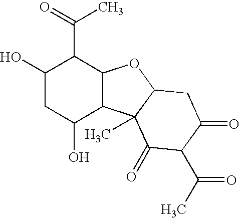

and the compound N-{4-[2,5-Dioxo-1-(4-trifluoromethoxy-phenyl)-pyrrolidin-3-yl]-phenyl}-2,2,2-trifluoro-acetamide (also known as Maybridge 15091, available from Maybridge PLC, England):

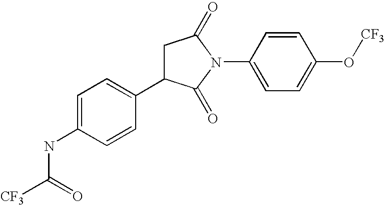

As stated above, the compounds of this invention having formula (I) or (II) may be substituted at one or more positions by substituted or unsubstituted aliphatic and/or heteroaliphatic groups. The terms “aliphatic” and “heteroaliphatic” are intended to be broadly construed.

The term “aliphatic,” means, unless otherwise stated, a non-aromatic, straight or branched chain, or cyclic, hydrocarbon moiety, either saturated or mono- or poly-unsaturated, including such a moiety that contains both cyclical and chain elements, having the designated number of carbon atoms (i.e. C₁-C₁₀ means one to ten carbon atoms). Types of saturated aliphatic hydrocarbon moieties include, e.g., alkyl, alkylene, cycloalkyl or cycloalkyl-alkyl groups, such as methyl, ethyl, n-propyl, isopropyl, n-butyl, t-butyl, isobutyl, sec-butyl, methylene, ethylene, n-butylene, cyclopropyl, cyclobutyl, cyclohexyl, cyclohexylmethyl, and cyclopropylmethyl, including homologs and isomers thereof, for example, n-pentyl, isopentyl, neopentyl, and the like.

An unsaturated aliphatic group may similarly be a cyclic or an acyclic group, and has one or more double and/or triple bonds. Examples of unsaturated aliphatic groups include vinyl, isoprenyl, 2-propenyl, crotyl, 2-isopentenyl, 2-(butadienyl), 2,4-pentadienyl, 3-(1,4-pentadienyl), ethynyl, 1- and 3-propynyl, 3-butynyl, cyclopentenyl, cyclohexenyl, cyclohexadienyl, and the like.

A “C₁-C₂₀ aliphatic group (or moiety)” is thus a substituted or unsubstituted aliphatic group having from 1 to 20 carbons. Similarly, a “C₁₁ aliphatic group” is a substituted or unsubstituted aliphatic group having 11 carbons. Both terms include cyclic and acyclic, and saturated and unsaturated groups.

A “lower aliphatic” or “lower alkylene” group is a shorter chain aliphatic or alkylene group, generally having eight or fewer carbon atoms.

The terms “oxyaliphatic”, “aminoaliphatic” and “thioaliphatic” are used in their conventional sense, and refer to aliphatic groups (as defined above) attached to the remainder of the molecule via an oxygen atom, an amino group, or a sulfur atom, respectively.

The term “heteroaliphatic,” by itself or in combination with another term, means, unless otherwise stated, a non-aromatic straight or branched chain, or cyclic, moiety, either saturated or mono- or polyunsaturated, including a moiety containing both cyclical and chain components, consisting of the stated number of carbon atoms and from one to four heteroatoms selected from the group consisting of O, N, Si and S, and wherein nitrogen and sulfur atoms may optionally be oxidized and/or nitrogen atoms may optionally be quaternary. When the moiety contains multiple heteroatoms, they may be the same or different. The heteroatom(s) may be placed at any position of the heteroaliphatic moiety. Examples include —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH₂—CH₂—S—CH₂CH₂—, —CH₂—S—CH₂—CH₂—NH—CH₂—, —OCH₂—, —OCH₂O— and —CH═CH—N(CH₃)—CH₃. Up to two heteroatoms may be consecutive, such as, for example, —CH₂—NH—OCH₃.

The terms “cycloaliphatic” and “heterocycloaliphatic”, by themselves or in combination with other terms, represent, unless otherwise stated, cyclic versions of “aliphatic” and “heteroaliphatic”, respectively. Additionally, for heterocycloaliphatic moieties, a heteroatom can occupy the position at which the heterocycle is attached to the remainder of the molecule. Examples of cycloaliphatic groups include cyclopentyl, cyclohexyl, 1-cyclohexenyl, 3-cyclohexenyl, cycloheptyl, and the like. Examples of heterocycloaliphatic groups include 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloaliphatic,” or “haloaryl” are meant to include such groups in which the halogens may be the same or different. For example, the term “halo(C₁-C₄) aliphatic” includes trifluoromethyl, difluorochloromethyl, 2,2,2-trifluoroethyl, 4-chlorobutyl, 3-bromopropyl, and the like. “Haloaryl” includes, for instance, mono-, di-, tri-, tetra-, and pentachlorophenyl, as well as groups having mixed substitutions such as 2-chloro-4-bromophenyl, etc,

The term “aryl” means, unless otherwise stated, a polyunsaturated aromatic hydrocarbon moiety, which can be a single ring or include multiple rings (up to three rings) fused together or linked covalently. Preferably, aryl groups contain a single ring. The term “heteroaryl” refers to aryl groups (or rings) that contain from zero to four heteroatoms selected from N, O, and S, wherein the nitrogen and sulfur atoms are optionally oxidized, and the nitrogen atom(s) are optionally quaternary. A heteroaryl group can be attached to the remainder of the molecule through a heteroatom. Some examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. The aryl groups in compounds of this invention may be substituted by substituents as described below.

For brevity, the term “aryl” when used in combination with other terms (e.g., aryloxy, arylthioxy, arylalkyl) includes both aryl and heteroaryl rings as defined above. Thus, the term “arylaliphatic” is meant to include those groups in which an aryl group is attached to or is a substituent on an aliphatic group, e.g., benzyl, phenethyl, pyridylmethyl and the like, including those aliphatic groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “aliphatic,” “heteroaliphatic,” “aryl” and “heteroaryl”) are meant to include both substituted and unsubstituted forms of the indicated moiety.

Substituents for the aliphatic, heteroaliphatic and aryl moieties, as well as for carbon or hetero atoms included within side rings D and E, can be a variety of groups, including but not limited to: —OR′, ═O, ═S, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NRR′R″)═NR′″, —NR′C(NR′R″)═NR′″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such group. R′, R″ and R′″ each independently refer to hydrogen, halogen, acyl (e.g., —C(O)CH₃, —C(O)CF₃, —C(O)CH₂OCH₃, and the like), optionally substituted heteroaliphatic, unsubstituted aryl, aryl substituted with 1-3 halogens, substituted or unsubstituted aliphatic, oxyaliphatic or thioaliphatic groups, or aryl-(C₁-C₄)aliphatic groups. When a compound of the invention includes more than one R group, each of the R groups is independently selected as are each R′, R″ and R′″ groups when more than one of these groups is present. When R′ and R″ are attached to the same nitrogen atom, they can be combined with the nitrogen atom and optionally an additional heteroatom to form a 5-, 6-, or 7-membered ring. For example, —NR′R″ is meant to include groups such as 1-pyrrolidinyl and 4-morpholinyl. From the above discussion of substituents, one skilled in the art will understand that the term “aliphatic”0 is meant to include groups such as haloaliphatic (e.g., —CF₃, —CHF₂ and —CH₂CF₃).

Two of the substituents on adjacent atoms of an aliphatic, heteroaliphatic, or aryl ring may optionally be replaced with a substituent of the formula —T—C(O)—(CRR′₂)ᵥ—U—, wherein T and U are independently —NR—, —O—, —CRR′— or a single bond, and v is an integer from 0 to 3. Alternatively, two of the substituents on adjacent atoms of the aryl or heteroaryl ring may optionally be replaced with a substituent of the formula —A—(CH₂)_{w}—B—, wherein A and B are independently —CRR′—, —O—, —NR—, —S—, —S(O)—, —S(O)₂—, —S(O)₂NR′— or a single bond, and w is an integer from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)_{y}—X—(CR″R′″)_{z}—, where y and z are independently integers from 0 to 3, and X is —O—, —NR′—, —S—, —S(O)—, —S(O)₂—, or —S(O)₂NR′—. The substituents R, R′, R″ and R′″ are independently selected from hydrogen or unsubstituted (C₁-C₆) alkyl. A typical substituent of the type described, on aromatic or heterocyclic rings, in the alkylenedioxy group, —O—(CH₂)ₘ—O, where m is an integer from 1 to 4, such as methylenedioxy, —OCH₂O— and ethylenedioxy, —OCH₂CH₂O—.

The term “pharmaceutically acceptable salts” is meant to include salts of the compounds in question that are prepared with relatively nontoxic acids or bases, depending on the particular substituents found on the compounds described herein. When compounds of the present invention contain relatively acidic functionalities, salts can be obtained by addition of the desired base, either neat or in a suitable inert solvent. Examples of pharmaceutically acceptable base addition salts include sodium, potassium, calcium, ammonium, organic amino, or magnesium salts, or the like. When compounds of the present invention contain relatively basic functionalities, salts can be obtained by addition of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galacturonic acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

Thus, for compounds of this invention, a group R or R₁-R₉ (when other than hydrogen) generally will comprise an aliphatic or heteroaliphatic group which may be substituted by one or more of the substituents mentioned above, including aryl groups as well as other aliphatic or cycloaliphatic groups (which themselves may be substituted by various substituents, for example, alkyl, haloalkyl, alkylol, alkoxy, alkylthio, hydroxy, halo, nitro, cyano, and the like).

The term “pharmacologically effective inhibitory amount” refers to an amount of a compound of the invention that will posses a desired inhibitory effect (as defined above) without demonstrating undue adverse effects on the subject or on cells or cell cultures being treated.

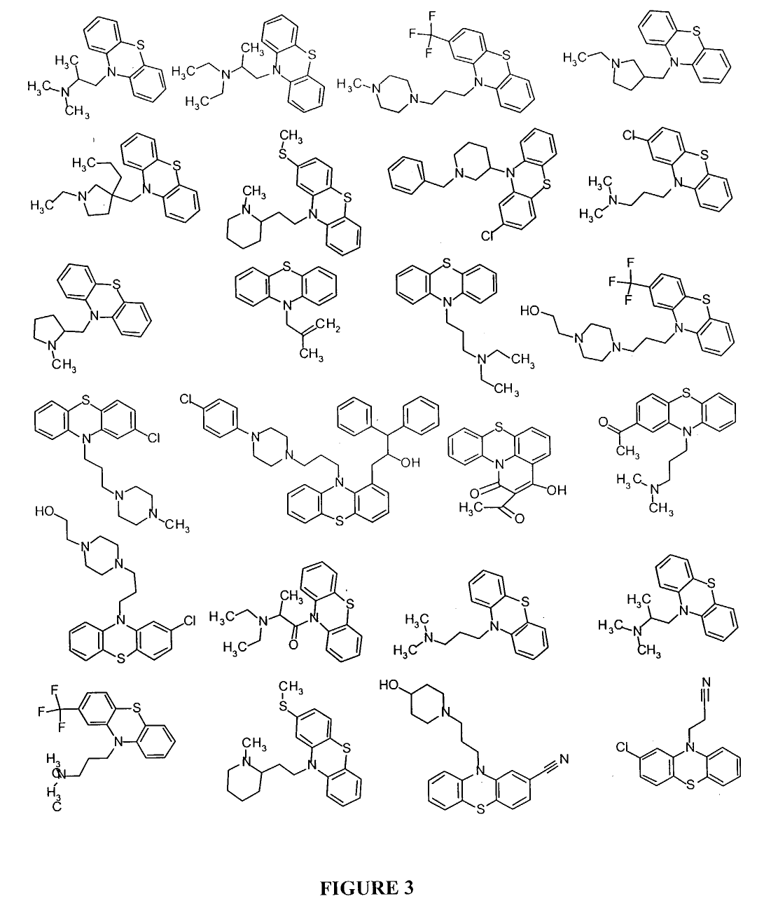

FIG. 3 depicts a number of phenothiazine compounds of the invention, showing various aliphatic and heteroaliphatic substituents on the nitrogen ring atom and on side ring atoms. In this series of compounds, for N-substituents (R) that contain a relatively bulky group such as a tertiary alkyl, optionally substituted phenyl, cycloaliphatic or cycloheteroaliphatic, such bulky substituent may be spaced from the ring by an alkylene group of at least one carbon atom.

The compounds in FIG. 3 represent members of a class of phenothiazines having the formula (II)

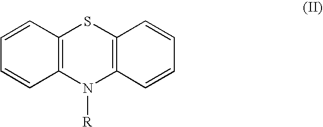

in which

R is hydrogen or an optionally substituted aliphatic, heteroaliphatic or cycloheteroaliphatic group, or in which the nitrogen atom, R, and two carbon atoms on the ring adjacent to the nitrogen atom, together comprise a ring having the formula —N—C═C—C(OH)═C(COCH₃)—C(O)—;

and in which carbon atoms on the side rings of the compound are optionally substituted by one or more of —OR′, ═O, ═S, ═NR′, ═N—OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NRR′R″)═NR′″, —NR′C(NR′R″)═NR′″,—NR—C(NR′R″)═NR′″, —S(O)R′, —S(O)₂R′, —S(O)₂NR′R″, —NRSO₂R′, —CN and/or —NO₂, in which R′, R″ and R′″ are each independently selected from hydrogen, halogen, acyl, optionally substituted heteroaliphatic groups, unsubstituted aryl, aryl substituted with 1-3 halogens, optionally substituted aliphatic, optionally substituted oxyaliphatic groups, optionally substituted thioaliphatic groups, or aryl- (C₁-C₄)aliphatic groups.;

and pharmaceutically acceptable salts thereof

As can be seen from FIG. 3, substituents on the central core structure can include halogen (preferably chloro), dialkylaminoalkyl (e.g., 2-[N,N-dimethylamino]propyl), haloalkyl (e.g., trifluoromethyl), alkyl-substituted heterocycloaliphatic-alkyl groups (e.g., N-ethyl-pyrrolidin-3-ylmethyl), alkylthio (e.g., methylthio), arylalkyl-heterocycloaliphatic (e.g., N-benzyl-piperidin-3-yl), alkenyl (e.g., isoprenyl), acyl (e.g., acetyl), acyl substituted by diaminoalkyl, cyano and cyanoalkyl (e.g., 2-cyanoethyl). Preferred members of this group are those in which the side rings are either unsubstituted or are substituted at the 2-position by halogen (most preferably chloro), trifluoromethyl, thiomethyl, acetyl or cyano.

Other preferred members of the group are those in which R is a C₂-C₄ alkylene group substituted by

(a) a mono-or dialkylamino group (in which the alkyl groups preferably independently have from 1 to 4 carbon atoms each), or by

(b) an optionally substituted cycloheteroaliphatic group [preferably saturated, and preferably one in which the ring of the cycloheteroaliphatic group contains from 5 to 7 atoms including 1-2 nitrogen atoms, and in which optional substituents on the ring are selected from C₁-C₄ alkyl, hydroxy, hydroxy-(C₁-C₃)alkyl, cyano-(C₁-C₃)alkyl and halophenyl],

or in which R is a C₂-C₄ unsaturated acyclic aliphatic group, optionally substituted, or in which R is a C₂-C₄ acyl group substituted by a mono-or dialkylamino group (preferably one in which the alkyl groups independently have from 1 to 4 carbon atoms each).

The compounds of FIG. 3 are known but, for the most part have not heretofore been known to possess pharmaceutical or pharmacological properties. Thus, pharmaceutical compositions containing pharmacologically effective amounts of those compounds known but not known to have such properties, form an aspect of this invention.

Some novel compounds of this invention have the formula (III):

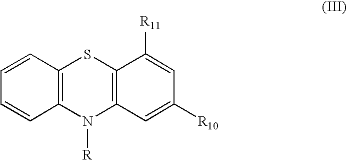

in which R is (CH₂)₃R₁₂; R₁₀ is halogen or C₁-C₄ alkoxy; R₁₁ is hydrogen if R₁₀ is halogen and is hydrogen or methyl if R₁₀ is alkoxy; and R₁₂ is selected from —N(CH₂CH₂)OH, —N(n-C₄H₉), —N(CH₂C₆H₅),

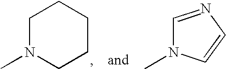

if R₁₀ is halogen, and is —N(CH₃)₂ if R₁₀ is alkoxy.

These may be made by the process described below.

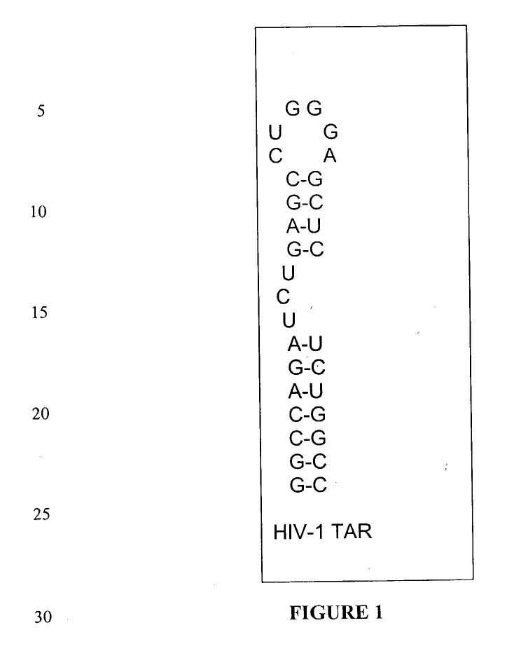

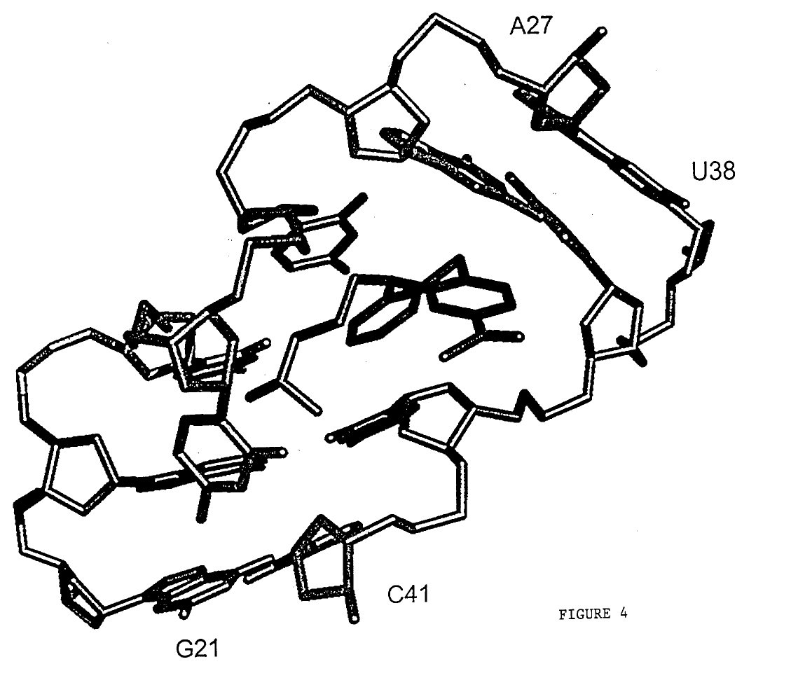

The compounds of this invention preferably have a central three-ring structure that is not completely planar. FIG. 1 shows the TAR (transactivation response element region of the RNA of the HIV genome. FIG. 4 for example, shows how the non-planar structure of acetylpromazine binds, and forms a complex, with the TAR. Other figures showing such binding are contained in the papers by Du et al., Structure of TAR RNA Complexed with a Nanomolecular Inhibitor of the Tat-TAR Interaction Identified by computational Screening Chemistry and Biology 9; 707 (2002) and Lind et al., Structure-based Computational Database Screening, In vitro Assay, and NMR Assessment of Compounds that Target TAR RNA, Chemistry & Biology 9, 185-193 (2002). The complete texts and figure of both publications are hereby fully incorporated herein.

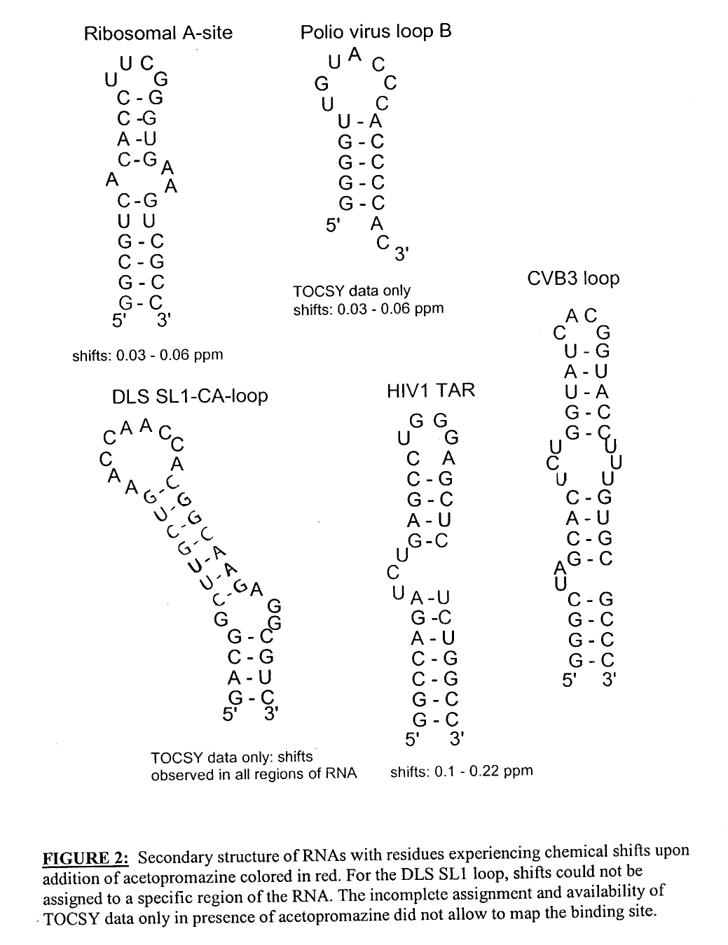

FIG. 2 shows bulge or loop regions of HIV TAR and four other RNAs—of the ribosomal A-site RNA, of the polio virus RNA [loop B], of the DLS SL-1 RNA (CA-loop), and of the CVB3 RNA. As described below, compounds of the invention have been found to bind to a bulge or loop in the regions depicted, and thus can inhibit functioning of the RNA and replication of the associated virus, or inhibit an associated microbial (e.g., bacterial) infection.

A number of compounds of this invention are commercially available from major chemical suppliers, but have not hitherto been known to be active in inhibiting replication of RNA or as antimicrobial or antiviral agents. Novel compounds of this invention may be prepared by various processes already known for making compounds of such classes, or by the process described below.

One process that may be used to prepare novel phenothiazines of this invention involves a three-step synthesis. The first step is a palladium-catalyzed condensation of anilines and aryl bromides. The next step is a thionation using elemental sulfur catalyzed by iodine. This surprisingly mild transformation will allow a wide variety of functionalities to be carried through to the phenothiazines. The final step is a condensation with an electrophile to alkylate the nitrogen, using, for instance, substituted alkyl bromides, which have been used extensively in the phenothiazine literature. Alternatively, condensation with carboxylic acids can be used to give amides.

Schematically, the process may be represented as follows:

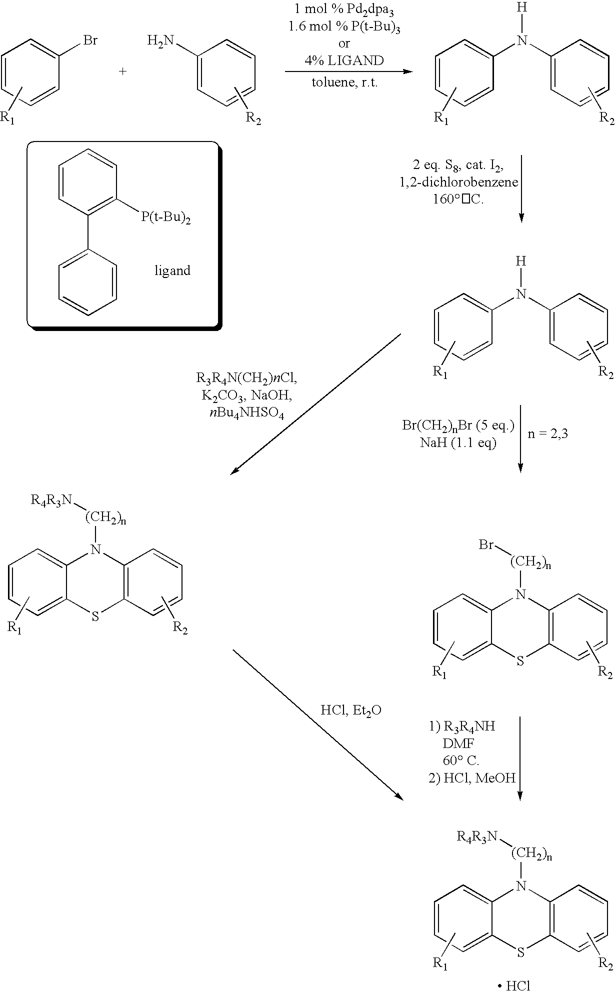

The process just described may be used either to prepare individual compounds or to prepare libraries of phenothiazines using conventional combinatorial chemistry techniques, for instance, using Bohdan reactor blocks on the 50 μM scale. This format affords a 96× degree of parallelization (in two blocks), thus allowing synthesis of the expected library sizes in 8 blocks. In such work, the reactions are worked up in situ in the blocks and solid phase extraction (in parallel), scavenger resins, and manual filtration are used to clean up the intermediates. The phenothiazine compounds thus prepared would be purified and isolated using, for instance, reverse phase HPLC.

For pharmaceutical use, the compounds of this invention are prepared and administered in the form of compositions or formulations. The compositions, which may be liquid or solid, will contain pharmaceutically acceptable diluents and/or carriers, i.e. diluents or carriers that are biocompatible and free from undesirable impurities.

The compositions may also be in the form of controlled release or sustained release compositions as known in the art, for instance, in matrices of biodegradable or non-biodegradable injectable polymeric microspheres or microcapsules, in liposomes, in emulsions, and the like.

For use, the compositions may be prepared in unit dosage forms that are sterilized and then placed within a container such as an ampoule. The compositions of this invention may, as stated above, be prepared in the form of single-dosage units for direct administration to a patient. However, more concentrated compositions may be prepared, from which the more dilute single-unit compositions may then be produced. The more concentrated compositions thus will contain substantially more than an inhibiting effective amount of the compound in question.

Thus, compounds of this invention can be formulated with a pharmaceutically acceptable carrier for administration to a subject. While any suitable carrier known to those of ordinary skill in the art may be employed in the pharmaceutical compositions of this invention, the type of carrier will vary depending on the mode of administration. The pharmaceutical composition is typically formulated such that the compound in question is present in a therapeutically effective amount, i.e., the amount of compound required to achieve the desired effect in terms of treating a subject.

For preparing pharmaceutical compositions, the pharmaceutically acceptable carriers can be either solid or liquid. Solid form preparations include powders, tablets, pills, capsules, cachets, suppositories, and dispersible granules. A solid carrier can be one or more substances that may also act as diluents, flavoring agents, binders, preservatives, tablet disintegrating agents, or an encapsulating material.

In powders, the carrier is a finely divided solid that is in a mixture with the finely divided active component. In tablets, the active component is mixed with the carrier having the necessary binding properties in suitable proportions and compacted in the shape and size desired.

Suitable carriers for the solid compositions of this invention include, for instance, magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component, with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution. In certain embodiments, the pharmaceutical compositions are formulated in a stable emulsion formulation (e.g., a water-in-oil emulsion or an oil-in-water emulsion) or an aqueous formulation that preferably comprises one or more surfactants. Suitable surfactants well known to those skilled in the art may be used in such emulsions. In one embodiment, the composition comprising the compound in question is in the form of a micellar dispersion comprising at least one suitable surfactant. The surfactants useful in such micellar dispersions include phospholipids. Examples of phospholipids include: diacyl phosphatidyl glycerols, such as: dimyristoyl phosphatidyl glycerol (DPMG), dipalmitoyl phosphatidyl glycerol (DPPG), and distearoyl phosphatidyl glycerol (DSPG); diacyl phosphatidyl cholines, such as: dimyristoyl phosphatidylcholine (DPMC), dipalmitoyl phosphatidylcholine (DPPC), and distearoyl phosphatidylcholine (DSPC); diacyl phosphatidic acids, such as: dimyristoyl phosphatidic acid (DPMA), dipalmitoyl phosphatidic acid (DPPA), and distearoyl phosphatidic acid (DSPA); and diacyl phosphatidyl ethanolamines such as: dimyristoyl phosphatidyl ethanolamine (DPME), dipalmitoyl phosphatidyl ethanolamine (DPPE), and distearoyl phosphatidyl ethanolamine (DSPE). Other examples include, but are not limited to, derivatives of ethanolamine (such as phosphatidyl ethanolamine, as mentioned above, or cephalin), serine (such as phosphatidyl serine) and 3′-O-lysyl glycerol (such as 3′-O-lysyl-phosphatidyglycerol).

Aqueous solutions suitable for oral use can be prepared by dissolving the compound in water and adding suitable colorants, flavors, stabilizers, and thickening agents as desired. Aqueous suspensions suitable for oral use can be made by dispersing the finely divided compound in water with viscous material, such as natural or synthetic gums, resins, methylcellulose, sodium carboxymethylcellulose, and other well-known suspending agents.

Also included are solid form preparations that are intended to be converted, shortly before use, to liquid form preparations for oral administration. Such liquid forms include solutions, suspensions, and emulsions. These preparations may contain, in addition to the active compound, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Compositions of compounds of this invention that are already available commercially for other pharmacological uses may be used to treat subjects so as to inhibit RNA replication. Dosages may be similar to those currently used for other purposes. For instance, promazines are prescribed to psychotic patients on a chronic basis 2 or 4 times per day, with up to one gram per day being given. Doses about 10 times lower are given to people for antiemetic purposes. It should be noted, in addition, that some of the compounds are already available in the form of various mono- or di-acid salts, such as their hydrochlorides and maleates.

The compounds (in the form of their compositions) are administered to patients by the usual means known in the art, for example, by injection, infusion, infiltration, implantation, irrigation, intranasally, orally, and the like. For administration by injection and/or infiltration or infusion, the compositions or formulations according to the invention may be suspended or dissolved as known in the art in a vehicle suitable for injection and/or infiltration or infusion. Such vehicles include isotonic saline, buffered or unbuffered and the like. Depending on the intended use, they also may contain other ingredients, including other active ingredients, such as isotonicity agents, sodium chloride, pH modifiers, colorants, preservatives, antibodies, enzymes, antibiotics, antifungals, antivirals, other anti-infective agents, and/or diagnostic aids such as radio-opaque dyes, radiolabeled agents, and the like, as known in the art. However, the compositions of this invention may comprise no more than a simple solution or suspension of a compound or a pharmaceutically acceptable salt of a compound, in distilled water or saline.

Compounds and compositions according to this invention may be administered alone or in conjunction with other therapeutic agents, for example other therapeutic agents that are used to treat viral or microbial diseases or conditions, or other conditions in patients or subjects being treated for such diseases or conditions, for example HIV-related complications or opportunistic infections.

Compounds of the invention may be administered to patients for so-called “therapeutic” purposes, i.e. to treat a condition that has been observed in the patient, or for prophylactic purposes, i.e. aimed at preventing or minimizing a condition that is suspected or expected to exist but that has not yet become apparent or been identified.

Thus, in certain embodiments, a compound of the invention is administered to a patient, preferably a mammal, more preferably a human, as a preventative measure against a disease associated with an RNA-ligand interaction in vivo. As used herein, “prevention” or “preventing” refers to a reduction of the risk of acquiring a disease. In one embodiment, a compound is administered as a preventative measure to a patient. According to this embodiment, the patient can have a genetic predisposition to a disease, such as a family history of the disease, or a non-genetic predisposition to the disease. Accordingly, the compound can be used for the treatment of one manifestation of a disease and prevention of another.

As used herein, “treatment” or “treating” refers to an amelioration of a disease, or at least one discernible symptom thereof, or to an amelioration of at least one measurable physical parameter, not necessarily discernible by the patient. In another embodiment, “treatment” or “treating” refers to inhibiting the progression of a disease, either physically, e.g., stabilization of a discernible symptom, physiologically, e.g., stabilization of a physical parameter, or both. In yet another embodiment, “treatment” or “treating” refers to delaying the onset of a disease. However, the compounds of the invention are not limited to use in treating pr preventing diseases in subjects. They may be used wherever inhibition of function of an RNA molecule is desired, for example to control viral or microbial infections in laboratory environments, e.g. in cells or cell cultures, or may applied to inhibit function of individual RNA molecules as desired or needed.

The effective inhibitory dose of compounds of the present invention may be determined by in vitro or in vivo assays, e.g., assessing the effects of the compound on viral replication or microbial infection in tissue culture or viral growth or microbial infection in an animal. The amount of compound administered in a pharmacologically inhibitory dose is dependent upon the age, weight, kind of concurrent treatment and nature of the condition being treated. Similarly, the effective inhibitor amount of a compound of the invention on a particular RNA that is not necessarily linked to a viral or microbial infection can be determined using in vitro tests in which the RNA is contacted with various amounts of the test compound.

According to this invention, compounds as described herein, whether or not known to have pharmacological activity, can inhibit replication of HIV by binding to TAR and inhibiting the TAR-Tat interaction. As discussed above, interrupting the interaction between Tat and TAR has been found to block HIV-1 replication in infected cells.

Very generally, suitable dosage ranges for oral administration are generally about 0.001 milligram to about 200 milligrams of a compound (including a pharmaceutically acceptable salt thereof) per kilogram body weight per day. In specific preferred embodiments of the invention, the oral dose is about 0.01 milligram to about 100 milligrams per kilogram body weight per day, more preferably about 0.1 milligram to about 75 milligrams per kilogram body weight per day, more preferably about 0.5 milligram to 5 milligrams per kilogram body weight per day. The dosage amounts described herein refer to total amounts administered; that is, if more than one compound of the invention is administered, or if a compound is administered with another therapeutic agent, then the preferred dosages correspond to the total amount administered. Oral compositions preferably contain about 10% to about 95% active ingredient by weight.

Suitable dosage ranges for intravenous (i.v.) administration are about 0.01 milligram to about 100 milligrams per kilogram body weight per day, about 0.1 milligram to about 35 milligrams per kilogram body weight per day, and about 1 milligram to about 10 milligrams per kilogram body weight per day. Suitable dosage ranges for intranasal administration are generally about 0.01 pg/kg body weight per day to about 1 mg/kg body weight per day. Suppositories generally contain about 0.01 milligram to about 50 milligrams of a compound of the invention per kilogram body weight per day and comprise active ingredient in the range of about 0.5% to about 10% by weight.

Recommended dosages for intradermal, intramuscular, intraperitoneal, subcutaneous, epidural, sublingual, intracerebral, intravaginal, transdermal administration or administration by inhalation are in the range of about 0.001 milligram to about 200 milligrams per kilogram of body weight per day. Suitable doses for topical administration are in the range of about 0.001 milligram to about 1 milligram, depending on the area of administration. Effective doses may be extrapolated from dose-response curves derived from in vitro or animal model test systems. Such animal models and systems are well known in the art.

As described below, based on an NMR-derived structural model of HIV-1 TAR, it was verified that at least one compound described herein (acetylpromazine) indeed binds to the 5′ bulge of TAR in solution. The 5′ bulge is the same region previously identified as the site of Tat binding. We have also shown that compounds of the invention, at concentrations between 0.1 and 1 μM, disrupt the Tat-TAR interaction in vitro.

Nine compounds of the invention have in this manner been found to bind to the TAR region. All are phenothiazines (A═S, B═N—R), and include acetopromazine (also known as acetylpromazine and acepromazine) {10-[3-(dimethylamino)-propyl)phenothiazin-2-yl methyl ketone}, chlorpromazine[2-chloro-10-(dimethylaminopropyl)phenothiazine] and prochlorperazine{3-chloro-10-[3-(4-methyl-1-piperazinyl-1′)propyl]-3-chlorophenothiazine}. Two others were not found to have such binding activity in the work conducted so far—trifluoperazine and thiethylperazine. However, trifluoperazine was found to demonstrate an inhibitory effect on another RNA—the RNA of the ribosomal A-site.

At a concentration of 100 nM, acetylpromazine completely inhibited the interaction of TAR and Tat, both at 100 nM.

Table 1 below summarizes the results of tests of six phenothiazines that are both known in general and are known to have certain pharmaceutical properties (but were not heretofore known as inhibitors of RNA function) (compounds 1-6 below), and five novel phenothiazines (compounds 7-11 below), on five types of RNA. The structures of these eleven compounds, and associated compounds numbers in Table 1, are given below.

Known Compounds

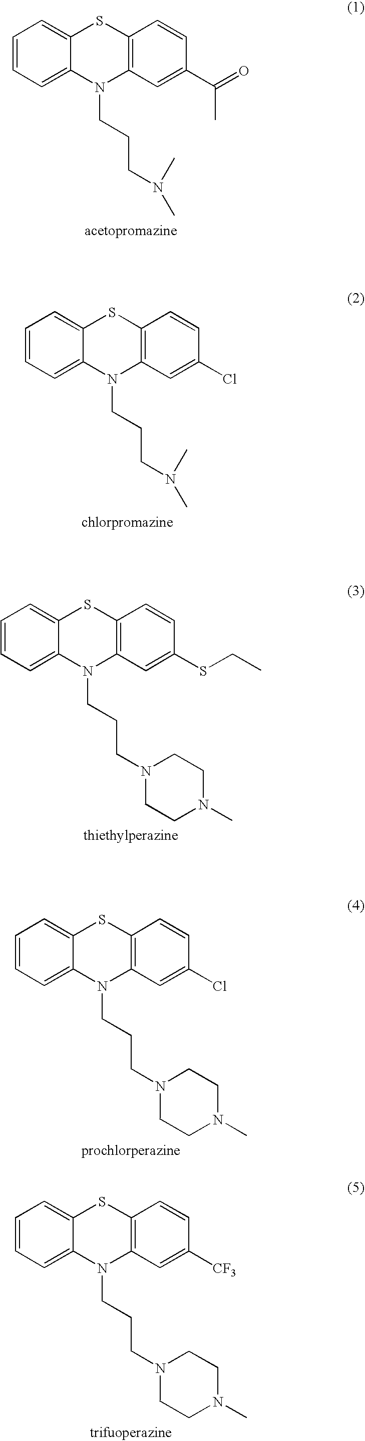

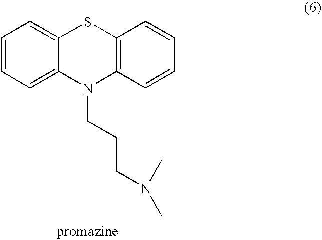

Synthesized Compounds

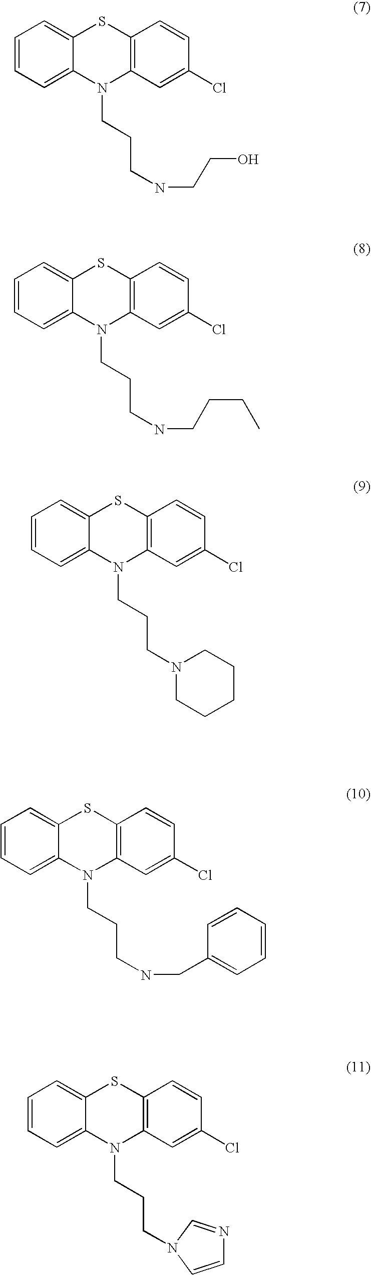

TABLE 1
PHENOTHIAZINE DERIVATIVES USED IN RNA BINDING EXPERIMENTS

| | | STD signals @ | | | |
|---|---|---|---|---|---|
| | | 0.4-0.6 | Gel Shift | TOCSY/NOESY | Line |
| RNA | DRUG | mM drug | Assay | data | broadening |
| Ribosomal | (1) | yes | | yes/yes | yes |
| A-site | (2) | yes | | no data/no data | yes |
| | (3) | no | | no data/no data | yes |
| | (4) | yes | | no data/no data | yes |
| | (5) | yes | | no data/no data | yes |
| | (6) | no | | no data/no data | little |
| HIV-1 TAR | (1) | yes | positive | yes/yes | yes |
| | (2) | no data | positive | | |
| | (3) | no data | negative | | |
| | (4) | no data | positive | | |
| | (5) | no data | negative | | |
| | (6) | no data | positive | | yes |
| | (7) | yes | | | yes |
| | (8) | yes | | | yes |
| | (9) | yes | | | little |
| | (10) | yes | | | little |
| | (11) | yes | | | |
| Polio virus | (1) | yes | | yes/no data | yes |
| loop B | | | | | |
| DLS SL1- | (1) | yes | | yes/no data | yes |
| CA-loop | | | | | |
| CVB3 loop | (1) | yes | | no data/no data | yes |
| D | | | | | |

The invention is further illustrated by the examples that follow. However, it should be noted that these are presented as examples of the invention, and do not limit it in any way.

### EXAMPLE 1

### Determination of the Structure of the Acetylpromazine-TAR Complex

Sample Preparation:

Five different samples of the 31-nucleotide TAR were prepared: unlabeled, uniformly ¹³C,¹⁵N-labeled, type-specifically ¹³C,¹⁵N-labeled at G, A or C residues, respectively. All samples were prepared by in vitro transcription using T7 RNA polymerase and a synthetic DNA template and purified as described in Du, et al., Biochemistry 35, 4187 (1996). Acetylpromazine was purchased from Research Diagnostics Inc. Final sample conditions were 1-2 mM RNA in 10 mM sodium phosphate buffer (pH 6.5), 20 mM sodium chloride. Acetylpromazine was added in about two-fold excess over RNA.

NMR Spectroscopy:

The solution structure of the acetylpromazine-TAR complex was determined using a protocol very similar to that previously described by Schmitz, U., et al, (1999) Structure of the phylogenetically most conserved domain of SRP RNA, RNA 5, 1419-1429.

All NMR experiments were performed on Varian Inova 600 MHz spectrometers. Spectra were processed with NMRpipe and analyzed with SPARKY. Homonuclear 2D NOESY spectra in H₂O were collected at 10° C. using the SSNOESY pulse sequence.

The structure of TAR with acetylpromazine bound is shown in FIG. 3. Due to the large number of intermolecular NOEs, 51, between acetylpromazine and TAR, binding of acetylpromazine to the 5′ bulge is very well defined.

The three-member ring of acetylpromazine inserts between base pairs G26-C39 and A22-U40, preventing continuous stacking of the lower and upper stems as observed in structures induced by argininamide or Tat-peptide. However, another set of base-stacking interactions is created. U23 continues stacking on A22 in the fashion of an A-helix. This is evident by the NOEs from U23 H6 to A22 H2′ and H3′, and from U23 H1′ to A22 H2. Benzene ring II of acetylpromazine is stacked on U40. Stacking of U23 on A22 and benzene ring II on U40 arranges the U23 base and benzene ring II such that they look like an extra base pair continuing helical stacking on the A22-U40 base pair. This “pseudo base-pair” may contribute to the creation of a deeper minor groove (compared to the standard A-helix) to accommodate the aliphatic moiety of acetylpromazine. On the major groove side, U25 stacks on benzene ring II of acetylpromazine.

The aliphatic moiety of acetylpromazine is extended along the minor groove. NOEs from the Hε protons of acetylpromazine to C41 H5 and H1′ protons are observed, indicating that the tail of the aliphatic chain is in close proximity to the G21-C41 base pair. C24 is also in the minor groove, with its base moiety close to the A 22-U40 and G21-C41 base pairs. C24 helps to bury the aliphatic chain of acetylpromazine within the minor groove.

### EXAMPLE 2

### RNA Binding Assays In vitro (Electrophoretic Mobility Shift Assay; EMSA)

EMSA is an effective method for determining whether TAR forms a complex with Tat and human cyclin T1. n the presence of TAR, containing the 5′ bulge and central loop, and these proteins, a lower mobility complex was observed on polyacrylamide gels. The lack of higher order complex formation signifies that a compound can block this RNA-protein interaction. Acetylpromazine, chlorpromazine and prochlorperazine, were found to prevent completely the binding between Tat and TAR at concentrations between 0.1 and 1 μM, with hybrid CycT1-Tat protein and TAR concentrations each at 0.1 μM, but two other phenothiazines tested—trifluoperazine and thioethylperazine—did not. These phenothiazines are used clinically as antipsychotic, sedative and antiemetic agents. Recent studies also suggest that they have antibiotic properties.

### EXAMPLE 3

### Chloramphenicol Acetyl Transferase (CAT) Assay

HeLa cells were pre-incubated with drug in concentrations ranging from 0.01-10 μM. Two different sets of targets and effectors were used. First, HIV-1 LTR and Tat were co-expressed. The heterologous tethering system of the Regulator of Expression of Virion genes (Rev) and its Rev response element (RRE) RNA were utilized as a control. If specific, a test compound should block Tat transactivation via TAR and not have any effects on the heterologous tethering of the RevTat fusion protein via RRE.

DNA constructs containing the engineered CAT gene preceded by TAR or RRE promoters were transfected into the HeLa cells with Lipofectin. Cells were incubated at 37° C., 5% CO₂ for 5 hours. Cells were rinsed, fresh drug and media (3 mL 10% Fetal Calf Serum, DMEM) added, and then incubated for 3 days at 37° C., 5% CO₂. Cells were collected by rinsing with ˜1 μL phosphate buffer and centrifugation for 4 minutes at 4000 rpm. Lysis buffer was added to the pellet, followed by centrifugation for 10 minutes at 14000 rpm. The supernatant was heated to 65° C. for 5 minutes (to remove background protein expression) and centrifuged 10 minutes at 14000 rpm. 100 μL of supernatant, 1 mg chloramphenicol, 1 μg ³H-acetyl-CoA, and EconoFluor solution were mixed, and immediately placed into the scintillation counter. CAT enzyme activity was measured by detecting the amount of ³H-acetyl-chloramphenicol.

At concentrations between 5 and 20 μM, prochlorperazine inhibited Tat transactivation up to 6-fold in a dose-dependent fashion. At the same time, this compound had no effect on the hybrid RevTat protein on the RRE. With prochlorperazine >20 μM, significant cellular toxicity resulted.

### EXAMPLE 4

### NMR Binding Experiments on other RNA Molecules

In order to detect the binding of the low molecular weight compounds to the RNA targets [RNA of the ribosomal A-site, of the polio virus, of the dimer linkage site stem-loop 1 (DLS SL1) of the HIV-1 virus, of the coxsackievirus B3 (CVB3) virus], we monitored three different effects indicative of the binding interactions: (a) line-broadening of the NMR signals of the small ligands, (b) chemical shift differences of both RNA and ligand signals in absence and presence of their respective binding partners, and (c) the observation of signals in saturation transfer difference (STD) NMR experiments.

a) Line-Broadening Effects:

Small molecules in non-viscous media have very sharp signals. Therefore, the broadening of NMR resonances of small molecules upon addition of a larger biomolecular target is an indication that the small molecule binds to the macromolecule. There are two effects contributing to the line-broadening, both of which are due to the reversible binding of the small molecule. The first contribution is due to the reduced molecular mobility of the larger target, which results in broader lines of large molecules. The small molecule, while bound to the target, has the same slow mobility and therefore, on average, has broader lines than non-binding compounds. However, this effect is not very pronounced for the small RNA molecules used here. The second contribution to the line-broadening originates from the chemical shift difference of the ligands signals between their free and bound states. This effect produces particularly broad lines for exchange processes that occur on an intermediate timescale relative to the NMR chemical shift differences Δδ. For fast and intermediate exchange processes one usually observes coalescence resulting in one signal occurring at the weighted average frequency. For slowly exchanging molecules, where the chemical shift difference is larger than the dissociation rate constant, one can observe two distinct resonances representing the free and bound signals, respectively. For most of the phenothiazines tested, we observed significant line broadening and shifting of the ligand signals upon addition of the respective RNAs. Although the observation of line broadening alone does not allow the classification into slow, intermediate or fast exchange, the data obtained suggests that these compounds are in fast to intermediate exchange, because we did not observe two distinct signals for any of the tested compounds, which would indicate a slow exchange process on the chemical shift timescale. This typically corresponds to K_{D} values ranging from ca. 10⁻³-10⁻⁶ [M] for the phenothiazines.

b) Chemical Shift Mapping:

By studying the changes in chemical shift of the RNA resonances in the presence and absence of ligands, we could determine which regions of the RNA were involved in the interaction process. For this, we needed to have the resonances of the RNA assigned in order to determine the RNA's binding epitope. Once the assignments are available, the simplest experiment to qualitatively map the binding site of the RNA is the TOCSY experiment, which allows monitoring of the shifts of the H5-H6 resonances of the pyrimidine bases. These experiments showed that acetylpromazine binds to specific tertiary RNA motifs such as loops and bulges, which are more accessible to binding interactions than regular double-stranded RNA stretches. We could not find any interactions of the phenothiazines with regular double-stranded or hairpin tetraloop RNA motifs.

c) Saturation Transfer Difference NMR Experiments:

Binding of a low molecular weight compound can be detected by so called Saturation Transfer Difference (STD) NMR experiments. These experiments detect the decrease of the binding molecule's signal intensity when the RNA or protein is selectively irradiated with a radiofrequency pulse. Since the binding interaction is detected directly, false positives are virtually eliminated. These one-dimensional STD NMR experiments can be used as a low-throughput screening method and have a high sensitivity compared to other NMR screening techniques. As with the above-mentioned line-broadening experiments, resonances of the RNA do not need to be assigned to observe the binding. Another advantage of this experiment is that direct characterization of binding molecules is attainable even from ligand mixtures because only the binding compound produces STD signals. In addition, differential STD signal intensities of the ligands can be used to gain information regarding the crucial parts of the ligand needed for interaction with the protein receptor. We also conducted a competition experiment where a mixture of acetylpromazine (1) and A-site RNA was titrated with the high affinity drug paromomycin (K_{D}=0.2 [μM]). From the reduction of STD signal intensities due to competition for the same binding site, we determined the IC₅₀. By applying the Cheng and Prusoff correction, we calculated a K_{D} value of ca. 10 μM for the interaction of acetylpromazine with A-site RNA. This value is in agreement with the intermediate exchange regime as inferred from the line-broadening experiments.

### EXAMPLE 5

### Synthesis of New Phenothiazines

The following experiments are representative of procedures carried out using the process route previously described. Designations of groups (R₁, R₂) refer to the process description rather than to the formulas (I)-(III) given herein for compounds of the invention.

N-(4-Tolyl)phenylamine (R₁=Me, R₂=H): To a solution of Pd₂dpa₃ (0.05 mmol, 45 mg) and P(t-Bu)₃ (800 μl, 0.1M solution in toluene) was added 4-bromotoluene (5 mmol, 855 mg), aniline (5 mmol, 456 μl) and sodium tert-butoxide (7.5 mmol, 720 mg) and the reaction mixture was stirred at rt for 18 h. The reaction mixture was treated with NH₄Cl (aq), diluted with ethylacetate and the organic phase was separated. The aqueous phase was extracted two times with ethylacetate and the combined organic layers were dried over MgSO₄, filtered and concentrated in vacuo to give 920 mg (99%) of 3 together with a small amount of the trialkylated amine as a brownish solid. ¹H-NMR (400 MHz) δ=7.23 (m, 2H), 7.07 (m, 2H), 7.00 (m, 4H), 6.87 (t, J=6.1 Hz, 1H), 5.57 (s, broad, NH), 2.29 (s, 3H); ¹³C-NMR (400 MHz) δ=143.9, 140.2, 130.9, 129.8, 129.3, 120.2, 118.9, 116.8, 20.6; Ms (EI) for C₁₃H₁₃N: m/z=.(M⁺); HRMS calcd for C₁₃H₁₃N 183.1047, found

7-Methylphenothiazine (R₁=Me, R₂=H): (1.5 mmol, 270 mg), S (3 mmol, 96 mg) and a catalytic amount of I₂ was dissolved in 1,2-dichlorobenzene (1 ml) and the reaction mixture was heated at 160° C. for 2 h. The crude reaction mixture was purified by column chromatography (SiO₂ ethylacetate/hexane, 1:14, R_{f}=0.18) gave 83 mg (28%) of 4 as a brownish solid. ¹H-NMR (400 MHz) δ=8.44 (s, NH), 6.97 (t, J=7.8 Hz, 1H), 6.89 (d, J=8.0 Hz, 1H), 6.78 (d, J=7.8 Hz, 1H), 6.74 (s, 1H), 6.72 (t, J=7.6 Hz, 1H), 6.65 (d, J=7.6 Hz, 1H), 6.58 (d, J=7.8 Hz, 1H), 3.32 (s, 3H); ¹³C-NMR (400 MHz) δ=; Ms (CI) for C₁₂H₉NS: m/z=???(M⁺); HRMS calcd for C₁₂H₉NS 199.0455, found ???.

10-(3-Bromopropyl)-2-chloro-phenothiazine (R=Cl, R₂=H)

To a mixture of 2-Chloro-10H-phenothiazine (250 mg), 1,3-dibromopropane (0.55 mL) and N, N-dimethylformamide (5 mL), sodium hydride (60% dispersion in mineral oil, 48 mg) was added under the ice-bath temperature. The reaction mixture was stirred at the room temperature for 1 hour, and additional sodium hydride ((60% dispersion in mineral oil, 48 mg) was added under the ice-bath temperature. The reaction mixture was stirred at the room temperature for 0.5 hour and worked up in a usual manner. The crude product was purified by a column chromatography (silica-gel; ethyl acetate % in n-hexane: 0% to 1.2% graduent) to give the title compound (170 mg). ¹H-NMR (CDCl₃): 2.32 (2H, quintet, J=6.4 Hz), 3.48 (2H, t, J=6.4 Hz), 4.06 (2H, t, J=6.4 Hz), 6.87-6.98 (4H, m), 7.05 (1H, d, J=8.4 Hz), 7.14-7.20 (2H, m) ppm

10-[3-(di- or monoalkylamino)propyl]-2-chloro-10H-phenothiazines (R₁=Cl, R₂=H; R₃=alkyl or H; R₄=alkyl or H)

10-(3-iodopropyl)-2-chloro-10H-phenothiazine (1.2 g) was dissolved with N,N-dimethylformamide; divided into five portions (0.24 g each); mixed with either n-butylamine (0.18 mL), benzylamine (0.20 mL), 2-aminoethanol (0.11 mL), piperidine (0.18 mL) or imidazole (120 mg); and heated at 60° C. for 19 hours. Each reaction mixture were worked up in a usual manner and purified by preparative thin-layer chromatography (silica-gel; dichloromethane: methanol: concentrated aqueous ammonia=90:10:0.1) to give the expected compounds. Each purified compound were dissolved with methanol (3 mL), and added 4N-hydrogen chloride in 1,4-dioxane (0.05 mL), evaporated to dryness and dissolved with 0.900 mL dimethylsulfoxide (biotech grade) to make 100 mM hydrochloride solution for the assay.

10-[2-(di- or monoalkylamino)ethyl]-2-chloro-10H-phenothiazines (R₁=Cl, R₂=H; R₃=alkyl or H; R₄=alkyl or H)

A mixture of 2-Chloro-10H-phenothiazine, 2-chloroethyl)-(mono or dialkylamine) hydrochloride (1.05 eq.), potassium carbonate (5 eq), ground sodium hydroxide (2 eq), tetra-n-butylammonium hydrogensulfate (34 mg) was stirred at ambient temperature for 8 h. The reaction mixture was worked up in a aqueously by extraction with ether and purified by preparative thin-layer chromatography and high-pressure liquid chromatography.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method for inhibition of RNA function comprising contacting an RNA molecule with a pharmacologically effective inhibitory amount of
(a) a compound having the formula (I): 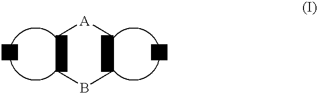
in which:
A is O; NR₁; S(O)ₙ where n is 0, 1 or 2; CR₂R₃; or —A′—CR₄R₅ where A′ is O, NR₁, S(O)ₙ wherein n is 0, 1 or 2 or CR₆R₇, wherein R₁ through R₇ are independently hydrogen or optionally substituted aliphatic or heteroaliphatic groups;
B is N—R, O, S or CR₈R₉ in which R is hydrogen or an optionally substituted aliphatic or heteroaliphatic group and R₈ and R₉ are independently hydrogen or optionally substituted aliphatic or heteroaliphatic groups; or when B is N—R, then R, the nitrogen atom of B, and two carbon atoms on the ring adjacent to the nitrogen atom together comprise a ring having the formula —N—C═C—C(OH)═C(COCH₃)—C(O)—;
provided that at least one of A and B comprises an oxygen, sulfur or nitrogen ring atom;
D and E, together with the carbon atoms to which they are bonded, independently comprise optionally substituted rings of 5-7 atoms selected from C, N, S and O, wherein each ring includes at least one double bond;
and pharmaceutically acceptable salts thereof;
(b) yohimbine;
(c) usnic acid; or
(d) N-{4-[2,5-Dioxo-1-(4-trifluoromethoxy-phenyl)-pyrrolidin-3-yl]-phenyl}-2,2,2-trifluoro-acetamide.

2. A method according to claim 1. in which the compound is of formula (I).

3. A method according to claim 2 in which D and E are unsubstituted rings.

4. A method according to claim 2 in which one of D and E is a mono-substituted ring.

5. A method according to claim 4 in which in which one of D and E is substituted by halogen, trifluoromethyl, cyano, C₁-C₄ alkoxy or acetyl.

6. A method according to claim 2 in which D and E are both mono-substituted rings.

7. A method according to claim 1. in which the compound has the formula (II) 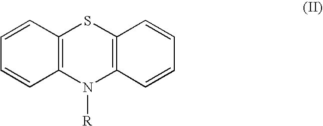
in which
R is hydrogen or an optionally substituted aliphatic, heteroaliphatic or cycloheteroaliphatic group, or in which the nitrogen atom, R, and two carbon atoms on the ring adjacent to the nitrogen atom, together comprise a ring having the formula —N—C═C—C(OH)═C(COCH₃)—C(O)—;
and in which carbon atoms on the side rings of the compound are optionally substituted by one or more of —OR′, ═O, ═S, ═NR′, ═N—OR′, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NRR′R″)═NR′″, —R′C(NR′R″)═NR′″, —NR—C(NR′R″)═NR′″, —S(O)R′, —S(O)₂R′, —S(O)₂NR′R″, —NRSO₂R′, —CN and/or —NO₂, in which R′, R″ and R′″ are each independently selected from hydrogen, halogen, acyl, optionally substituted heteroaliphatic groups, unsubstituted aryl, aryl substituted with 1-3 halogens, optionally substituted aliphatic, optionally substituted oxyaliphatic groups, optionally substituted thioaliphatic groups, or aryl- (C₁-C₄)aliphatic groups.;
and pharmaceutically acceptable salts thereof.

8. A method according to claim 7 in which both side rings are unsubstituted rings.

9. A method according to claim 7 in which one or both of the side rings is a mono-substituted ring.

10. A method according to claim 9 in which in which one of the side rings is substituted by halogen, trifluoromethyl, cyano, C₁-C₄ alkoxy or acetyl.

11. A method according to claim 7 in which the side rings are both mono-substituted rings.

12. A method according to claim 7 in which the side rings are either unsubstituted or are substituted at the 2-position by halogen, trifluoromethyl, thiomethyl, acetyl, C₁-C₄ alkoxy or cyano, and in which R is
(a) a C₂-C₄ alkylene group substituted by a mono- or dialkylamino group or by an optionally substituted cycloheteroaliphatic group;
(b) an optionally substituted C₂-C₄ unsaturated acyclic aliphatic group, or
(c) a C₂-C₄ acyl group substituted by a mono- or dialkylamino group.

13. A method according to claim 7 in which the compound is acetylpromazine, chlorpromazine, prochlorperazine, promazine, or trifluoperazine.

14. A method according to claim 2. in which the compound has the formula (III): 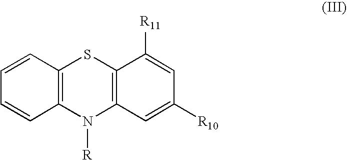
in which R is (CH₂)₃R₁₂; R₁₀ is halogen or C₁-C₄ alkoxy; R₁₁ is hydrogen if R₁₀ is halogen and is hydrogen or methyl if R₁₀ is alkoxy; and R₁₂ is selected from —N(CH₂CH₂)OH, —N(n-C₄H₉), —N(CH₂C₆H₅), 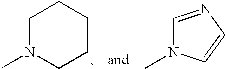
if R₁₀ is halogen, and is —N(CH₃)₂ if R₁₀ is alkoxy.

15. A method according to claim 1. in which the compound is yohimbine.

16. A method according to claim 1. in which the compound is usnic acid.

17. A method according to claim 1. in which the compound is N-{4-[2,5-Dioxo-1-(4-trifluoromethoxy-phenyl)-pyrrolidin-3-yl]-phenyl}-2,2,2-trifluoro-acetamide.

18. A method according to claim 1. in which the compound is a phenothiazine.

19. A method according to claim 1. in which the compound is a thioxanthene.

20. A method according to claim 1. in which the compound is a thianthrene.

21. A method according to claim 1. in which the compound is a phenoxazine.

22. A method according to claim 1. in which the compound is a phenazine.

23. A method according to claim 1. in which the compound is a phenoxathiin.

24. A method according to claim 1. in which the compound is a benzepine.

25. A method according to claim 1. in which the RNA is viral or microbial RNA.

26. A method according to claim 1. in which the RNA is viral RNA.

27. A method according to claim 1. in which the RNA is retroviral RNA.

28. A method according to claim 1. in which the RNA is HIV RNA.

29. A method according to claim 18 in which the compound inhibits RNA function by binding to the TAR site.

30. A method according to claim 1. in which the RNA is microbial RNA

31. A method according to claim 28 in which the RNA is bacterial RNA.

32. A method according to claim 28 in which the RNA is fungal RNA

33. A method according to claim 28 in which the RNA is protozoal RNA.

34. A method for inhibition of RNA function comprising contacting cells comprising RNA with a pharmacologically effective inhibitory amount of
(a) a compound having the formula (I): 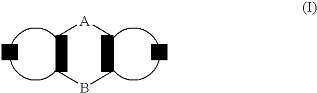
in which:
A is O; NR₁; S(O)ₙ where n is 0, 1 or 2; CR₂R₃; or —A′—CR₄R₅ where A′ is O, NR₁, S(O)ₙ wherein n is 0, 1 or 2 or CR₆R₇, wherein R₁ through R₇ are independently hydrogen or optionally substituted aliphatic or heteroaliphatic groups;
B is N—R, O, S or CR₈R₉ in which R is hydrogen or an optionally substituted aliphatic or heteroaliphatic group and R₈ and R₉ are independently hydrogen or optionally substituted aliphatic or heteroaliphatic groups; or when B is N—R, then R, the nitrogen atom of B, and two carbon atoms on the ring adjacent to the nitrogen atom together comprise a ring having the formula —N—C═C—C(OH)═C(COCH₃)—C(O)—;
provided that at least one of A and B comprises an oxygen, sulfur or nitrogen ring atom;
D and E, together with the carbon atoms to which they are bonded, independently comprise optionally substituted rings of 5-7 atoms selected from C, N, S and O, wherein each ring includes at least one double bond;
and pharmaceutically acceptable salts thereof;
(b) yohimbine;
(c) usnic acid; or
(d) N-{4-[2,5-Dioxo-1-(4-trifluoromethoxy-phenyl)-pyrrolidin-3-yl]-phenyl}-2,2,2-trifluoro-acetamide.

35. A method according to claim 34 in which the compound is of formula (I).

36. A method according to claim 35 in which D and E are unsubstituted rings.

37. A method according to claim 36 in which one of D and E is a mono-substituted ring.

38. A method according to claim 37 in which in which one of D and E is substituted by halogen, trifluoromethyl, cyano, C₁-C₄ alkoxy or acetyl.

39. A method according to claim 35 in which D and E are both mono-substituted rings.

40. A method according to claim 34 in which the compound has the formula (II) 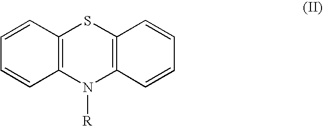
in which
R is hydrogen or an optionally substituted aliphatic, heteroaliphatic or cycloheteroaliphatic group, or in which the nitrogen atom, R, and two carbon atoms on the ring adjacent to the nitrogen atom, together comprise a ring having the formula —N—C═C—C(OH)═C(COCH₃)—C(O)—;
and in which carbon atoms on the side rings of the compound are optionally substituted by one or more of —OR′, ═O, ═S, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NRR′R″)═NR′″, —R′C(NR′R″)═NR′″, —NR—C(NR′R″)═NR′″, —S(O)R′, —S(O)₂R′, —S(O)₂NR′R″, —NRSO₂R′, —CN and/or —NO₂, in which R′, R″ and R′″ are each independently selected from hydrogen, halogen, acyl, optionally substituted heteroaliphatic groups, unsubstituted aryl, aryl substituted with 1-3 halogens, optionally substituted aliphatic, optionally substituted oxyaliphatic groups, optionally substituted thioaliphatic groups, or aryl- (C₁-C₄)aliphatic groups.;
and pharmaceutically acceptable salts thereof.

41. A method according to claim 40 in which both side rings are unsubstituted rings.

42. A method according to claim 40 in which one or both of the side rings is a mono-substituted ring.

43. A method according to claim 42 in which in which one of the side rings is substituted by halogen, trifluoromethyl, cyano, C₁-C₄ alkoxy or acetyl.

44. A method according to claim 40 in which the side rings are both mono-substituted rings.

45. A method according to claim 40 in which the side rings are either unsubstituted or are substituted at the 2-position by halogen, trifluoromethyl, thiomethyl, acetyl, C₁-C₄ alkoxy or cyano, and in which R is
(a) a C₂-C₄ alkylene group substituted by a mono- or dialkylamino group or by an optionally substituted cycloheteroaliphatic group;
(b) an optionally substituted C₂-C₄ unsaturated acyclic aliphatic group, or
(c) a C₂-C₄ acyl group substituted by a mono- or dialkylamino group.

46. A method according to claim 40 in which the compound is acetylpromazine, chlorpromazine, prochlorperazine, promazine, or trifluoperazine.

47. A method according to claim 35 in which the compound has the formula (III): 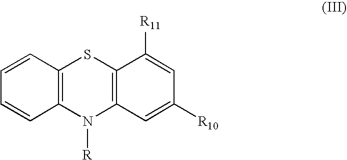
in which R is (CH₂)₃R₁₂; R₁₀ is halogen or C₁-C₄ alkoxy; R₁₁ is hydrogen if R₁₀ is halogen and is hydrogen or methyl if R₁₀ is alkoxy; and R₁₂ is selected from —N(CH₂CH₂)OH, —N(n-C₄H₉), —N(CH₂C₆H₅), 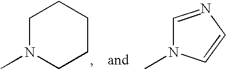
if R₁₀ is halogen, and is —N(CH₃)₂ if R₁₀ is alkoxy.

48. A method according to claim 34 in which the compound is yohimbine.

49. A method according to claim 34 in which the compound is usnic acid.

50. A method according to claim 34 in which the compound is N-{4-[2,5-Dioxo-1-(4-trifluoromethoxy-phenyl)-pyrrolidin-3-yl]-phenyl}-2,2,2-trifluoro-acetamide.

51. A method according to claim 34 in which the compound is a phenothiazine.

52. A method according to claim 34 in which the compound is a thioxanthene.

53. A method according to claim 34 in which the compound is a thianthrene.

54. A method according to claim 34 in which the compound is a phenoxazine.

55. A method according to claim 34 in which the compound is a phenazine.

56. A method according to claim 34 in which the compound is a phenoxathiin.

57. A method according to claim 34 in which the compound is a benzepine.

58. A method for inhibition of microbial infection in a subject comprising administering to said subject a pharmacologically effective inhibitory amount of
(a) a compound having the formula (I): 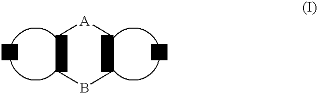
in which:
A is O; NR₁; S(O)ₙ where n is 0, 1 or 2; CR₂R₃; or —A′—CR₄R₅ where A′ is O, NR₁, S(O)ₙ wherein n is 0, 1 or 2 or CR₆R₇, wherein R₁ through R₇ are independently hydrogen or optionally substituted aliphatic or heteroaliphatic groups;
B is N—R, O, S or CR₈R₉ in which R is hydrogen or an optionally substituted aliphatic or heteroaliphatic group and R₈ and R₉ are independently hydrogen or optionally substituted aliphatic or heteroaliphatic groups; or when B is N—R, then R, the nitrogen atom of B, and two carbon atoms on the ring adjacent to the nitrogen atom together comprise a ring having the formula —N—C═C—C(OH)═C(COCH₃)—C(O)—;
provided that at least one of A and B comprises an oxygen, sulfur or nitrogen ring atom;
D and E, together with the carbon atoms to which they are bonded, independently comprise optionally substituted rings of 5-7 atoms selected from C, N, S and O, wherein each ring includes at least one double bond;
and pharmaceutically acceptable salts thereof;
(b) yohimbine;
(c) usnic acid; or
(d) N-{4-[2,5-Dioxo-1-(4-trifluoromethoxy-phenyl)-pyrrolidin-3-yl]-phenyl}-2,2,2-trifluoro-acetamide.

59. A method according to claim 58 in which the compound is of formula (I).

60. A method according to claim 59 in which D and E are unsubstituted rings.

61. A method according to claim 59 in which one of D and E is a mono-substituted ring.

62. A method according to claim 62 in which in which one of D and E is substituted by halogen, trifluoromethyl, cyano, C₁-C₄ alkoxy or acetyl.

63. A method according to claim 59 in which D and E are both mono-substituted rings.

64. A method according to claim 58 in which the compound has the formula (II) 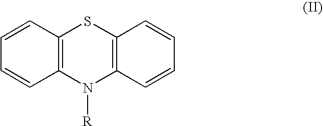
in which
R is hydrogen or an optionally substituted aliphatic, heteroaliphatic or cycloheteroaliphatic group, or in which the nitrogen atom, R, and two carbon atoms on the ring adjacent to the nitrogen atom, together comprise a ring having the formula —N—C═C—C(OH)═C(COCH₃)—C(O)—;
and in which carbon atoms on the side rings of the compound are optionally substituted by one or more of —OR′, ═O, ═S, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NRR′R″)═NR′″, —R′C(NR′R″)═NR′″, —NR—C(NR′R″)═NR′″, —S(O)R′, —S(O)₂R′, —S(O)₂NR′R″, —NRSO₂R′, —CN and/or —NO₂, in which R′, R″ and R′″ are each independently selected from hydrogen, halogen, acyl, optionally substituted heteroaliphatic groups, unsubstituted aryl, aryl substituted with 1-3 halogens, optionally substituted aliphatic, optionally substituted oxyaliphatic groups, optionally substituted thioaliphatic groups, or aryl- (C₁-C₄)aliphatic groups.;
and pharmaceutically acceptable salts thereof.

65. A method according to claim 64 in which both side rings are unsubstituted rings.

66. A method according to claim 64 in which one or both of the side rings is a mono-substituted ring.

67. A method according to claim 66 in which one of the side rings is substituted by halogen, trifluoromethyl, cyano, C₁-C₄ alkoxy or acetyl.

68. A method according to claim 64 in which the side rings are both mono-substituted rings.

69. A method according to claim 64 in which the side rings are either unsubstituted or are substituted at the 2-position by halogen, trifluoromethyl, thiomethyl, acetyl, C₁-C₄ alkoxy or cyano, and in which R is
(a) a C₂-C₄ alkylene group substituted by a mono- or dialkylamino group or by an optionally substituted cycloheteroaliphatic group;
(b) an optionally substituted C₂-C₄ unsaturated acyclic aliphatic group, or
(c) a C₂-C₄ acyl group substituted by a mono- or dialkylamino group.

70. A method according to claim 64 in which the compound is acetylpromazine, chlorpromazine, prochlorperazine, promazine, or trifluoperazine.

71. A method according to claim 59 in which the compound has the formula (III): 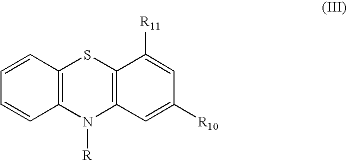
in which R is (CH₂)₃R₁₂; R₁₀ is halogen or C₁-C₄ alkoxy; R₁₁ is hydrogen if R₁₀ is halogen and is hydrogen or methyl if R₁₀ is alkoxy; and R₁₂ is selected from —N(CH₂CH₂)OH, —N(n-C₄H₉), —N(CH₂C₆H₅), 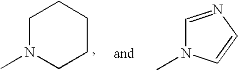
if R₁₀ is halogen, and is —N(CH₃) ₂ if R₁₀ is alkoxy.

72. A method according to claim 58 in which the compound is yohimbine.

73. A method according to claim 58 in which the compound is usnic acid.

74. A method according to claim 58 in which the compound is N-{4-[2,5-Dioxo-1-(4-trifluoromethoxy-phenyl)-pyrrolidin-3-yl]-phenyl}-2,2,2-trifluoro-acetamide.

75. A method according to claim 58 in which the compound is a phenothiazine.

76. A method according to claim 58 in which the compound is a thioxanthene.

77. A method according to claim 58 in which the compound is a thianthrene.

78. A method according to claim 58 in which the compound is a phenoxazine.

79. A method according to claim 58 in which the compound is a phenazine.

80. A method according to claim 58 in which the compound is a phenoxathiin.

81. A method according to claim 58 in which the compound is a benzepine.

82. A method according to claim 65 in which the infection is a bacterial infection.

83. A method for inhibiting a viral infection in a subject comprising administering to said subject a pharmacologically effective inhibitory amount of
(a) a compound having the formula (I): 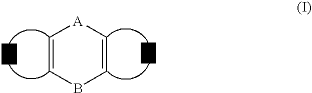
in which:
A is O; NR₁; S(O)ₙ where n is 0, 1 or 2; CR₂R₃; or —A′—CR₄R₅ where A′ is O, NR₁, S(O)ₙ wherein n is 0, 1 or 2 or CR₆R₇, wherein R₁ through R₇ are independently hydrogen or optionally substituted aliphatic or heteroaliphatic groups;
B is N—R, O, S or CR₈R₉ in which R is hydrogen or an optionally substituted aliphatic or heteroaliphatic group and R₈ and R₉ are independently hydrogen or optionally substituted aliphatic or heteroaliphatic groups; or when B is N—R, then R, the nitrogen atom of B, and two carbon atoms on the ring adjacent to the nitrogen atom together comprise a ring having the formula —N—C═C—C(OH)═C(COCH₃)—C(O)—;
provided that at least one of A and B comprises an oxygen, sulfur or nitrogen ring atom;
D and E, together with the carbon atoms to which they are bonded, independently comprise optionally substituted rings of 5-7 atoms selected from C, N, S and O, wherein each ring includes at least one double bond;
and pharmaceutically acceptable salts thereof;
(b) yohimbine;
(c) usnic acid; or
(d) N-{4-[2,5-Dioxo-1-(4-trifluoromethoxy-phenyl)-pyrrolidin-3-yl]-phenyl}-2,2,2-trifluoro-acetamide.

84. A method according to claim 83 in which the compound is of formula (I).

85. A method according to claim 84. in which D and E are unsubstituted rings.

86. A method according to claim 84. in which one of D and E is a mono-substituted ring.

87. A method according to claim 86 in which in which one of D and E is substituted by halogen, trifluoromethyl, cyano, C₁-C₄ alkoxy or acetyl.

88. A method according to claim 84 in which D and E are both mono-substituted rings.

89. A method according to claim 83 in which the compound has the formula (II) 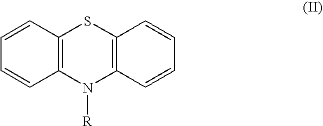
in which
R is hydrogen or an optionally substituted aliphatic, heteroaliphatic or cycloheteroaliphatic group, or in which the nitrogen atom, R, and two carbon atoms on the ring adjacent to the nitrogen atom, together comprise a ring having the formula —N—C═C—C(OH)═C(COCH₃)—C(O)—;
and in which carbon atoms on the side rings of the compound are optionally substituted by one or more of —OR′, ═O, ═S, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NRR′R″)═NR′″, —R′C(NR′R″)═NR′″, —NR—C(NR′R″)═NR′″, —S(O)R′, —S(O)₂R′, —S(O)₂NR′R″, —NRSO₂R′, —CN and/or —NO₂, in which R′, R″ and R′″ are each independently selected from hydrogen, halogen, acyl, optionally substituted heteroaliphatic groups, unsubstituted aryl, aryl substituted with 1-3 halogens, optionally substituted aliphatic, optionally substituted oxyaliphatic groups, optionally substituted thioaliphatic groups, or aryl- (C₁-C₄)aliphatic groups.;
and pharmaceutically acceptable salts thereof.

90. A method according to claim 89 in which both side rings are unsubstituted rings.

91. A method according to claim 89 in which one or both of the side rings is a mono-substituted ring.

92. A method according to claim 91 in which in which one of the side rings is substituted by halogen, trifluoromethyl, cyano, C₁-C₄ alkoxy or acetyl.

93. A method according to claim 89 in which the side rings are both mono-substituted rings.

94. A method according to claim 89 in which the side rings are either unsubstituted or are substituted at the 2-position by halogen, trifluoromethyl, thiomethyl, acetyl, C₁-C₄ alkoxy or cyano, and in which R is
(a) a C₂-C₄ alkylene group substituted by a mono- or dialkylamino group or by an optionally substituted cycloheteroaliphatic group;
(b) an optionally substituted C₂-C₄ unsaturated acyclic aliphatic group, or
(c) a C₂-C₄ acyl group substituted by a mono- or dialkylamino group.

95. A method according to claim 89 in which the compound is acetylpromazine, chlorpromazine, prochlorperazine, promazine, or trifluoperazine.

96. A method according to claim 84 in which the compound has the formula (III): 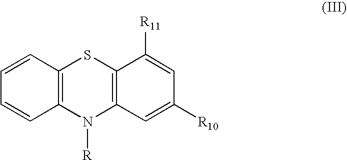
in which R is (CH₂)₃R₁₂; R₁₀ is halogen or C₁-C₄ alkoxy; R₁₁ is hydrogen if R₁₀ is halogen and is hydrogen or methyl if R₁₀ is alkoxy; and R₁₂ is selected from —N(CH₂CH₂)OH, —N(n-C₄H₉), —N(CH₂C₆H₅), 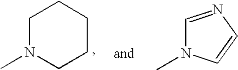
if R₁₀ is halogen, and is —N(CH₃)₂ if R₁₀ is alkoxy.

97. A method according to claim 83 in which the compound is yohimbine.

98. A method according to claim 83 in which the compound is usnic acid.

99. A method according to claim 83 in which the compound is N-{4-[2,5-Dioxo-1-(4-trifluoromethoxy-phenyl)-pyrrolidin-3-yl]-phenyl}-2,2,2-trifluoro-acetamide.

100. A method according to claim 83 in which the compound is a phenothiazine.

101. A method according to claim 83 in which the compound is a thioxanthene.

102. A method according to claim 83 in which the compound is a thianthrene.

103. A method according to claim 83 in which the compound is a phenoxazine.

104. A method according to claim 83 in which the compound is a phenazine.

105. A method according to claim 83 in which the compound is a phenoxathiin.

106. A method according to claim 83 in which the compound is a benzepine.

107. A method according to claim 110 wherein the viral infection comprises a retroviral infection.

108. A method according to claim 110 wherein the viral infection comprises AIDS.

109. A method according to claim 110 wherein the viral infection comprises a polio viral infection.

110. A method according to claim 110 wherein the viral infection comprises a rhinoviral infection.

111. A method according to claim 34 wherein the viral infection comprises an enteroviral infection.

112. A method according to claim 34 wherein the viral infection comprises hepatitis C.

113. A method for inhibition of viral RNA function comprising administering to a subject a pharmacologically effective inhibitory amount of
(a) a compound having the formula (I): 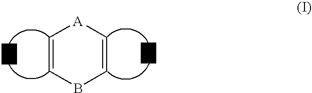
in which:
A is O; NR₁; S(O)ₙ where n is 0, 1 or 2; CR₂R₃; or —A′—CR₄R₅ where A′ is O, NR₁, S(O)ₙ wherein n is 0, 1 or 2 or CR₆R₇, wherein R₁ through R₇ are independently hydrogen or optionally substituted aliphatic or heteroaliphatic groups;
B is N—R, O, S or CR₈R₉ in which R is hydrogen or an optionally substituted aliphatic or heteroaliphatic group and R₈ and R₉ are independently hydrogen or optionally substituted aliphatic or heteroaliphatic groups; or when B is N—R, then R, the nitrogen atom of B, and two carbon atoms on the ring adjacent to the nitrogen atom together comprise a ring having the formula —N—C═C—C(OH)═C(COCH₃)—C(O)—;
provided that at least one of A and B comprises an oxygen, sulfur or nitrogen ring atom;
D and E, together with the carbon atoms to which they are bonded, independently comprise optionally substituted rings of 5-7 atoms selected from C, N, S and O, wherein each ring includes at least one double bond;
and pharmaceutically acceptable salts thereof;
(b) yohimbine;
(c) usnic acid; or
(d) N-{4-[2,5-Dioxo-1-(4-trifluoromethoxy-phenyl)-pyrrolidin-3-yl]-phenyl}-2,2,2-trifluoro-acetamide.

114. A method according to claim 113 in which the compound is of formula (I).

115. A method according to claim 114 in which D and E are unsubstituted rings.

116. A method according to claim 114 in which one of D and E is a mono-substituted ring.

117. A method according to claim 116 in which in which one of D and E is substituted by halogen, trifluoromethyl, cyano, C₁-C₄ alkoxy or acetyl.

118. A method according to claim 114 in which D and E are both mono-substituted rings.

119. A method according to claim 113 in which the compound has the formula (II) 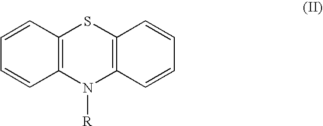
in which
R is hydrogen or an optionally substituted aliphatic, heteroaliphatic or cycloheteroaliphatic group, or in which the nitrogen atom, R, and two carbon atoms on the ring adjacent to the nitrogen atom, together comprise a ring having the formula —N—C═C—C(OH)═C(COCH₃)—C(O)—;
and in which carbon atoms on the side rings of the compound are optionally substituted by one or more of —OR′, ═O, ═S, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NRR′R″)═NR′″, —R′C(NR′R″)═NR′″, —NR—C(NR′R″)═NR′″, —S(O)R′, —S(O)₂R′, —S(O)₂NR′R″, —NRSO₂R′, —CN and/or —NO₂, in which R′, R″ and R′″ are each independently selected from hydrogen, halogen, acyl, optionally substituted heteroaliphatic groups, unsubstituted aryl, aryl substituted with 1-3 halogens, optionally substituted aliphatic, optionally substituted oxyaliphatic groups, optionally substituted thioaliphatic groups, or aryl- (C₁-C₄)aliphatic groups.;
and pharmaceutically acceptable salts thereof.

120. A method according to claim 119 in which both side rings are unsubstituted rings.

121. A method according to claim 119 in which one or both of the side rings is a mono-substituted ring.

122. A method according to claim 119 in which one of the side rings is substituted by halogen, trifluoromethyl, cyano, C₁-C₄ alkoxy or acetyl.

123. A method according to claim 119 in which the side rings are both mono-substituted rings.

124. A method according to claim 119 in which the side rings are either unsubstituted or are substituted at the 2-position by halogen, trifluoromethyl, thiomethyl, acetyl, C₁-C₄ alkoxy or cyano, and in which R is
(a) a C₂-C₄ alkylene group substituted by a mono- or dialkylamino group or by an optionally substituted cycloheteroaliphatic group;
(b) an optionally substituted C₂-C₄ unsaturated acyclic aliphatic group, or
(c) a C₂-C₄ acyl group substituted by a mono- or dialkylamino group.

125. A method according to claim 119 in which the compound is acetylpromazine, chlorpromazine, prochlorperazine, promazine, or trifluoperazine.

126. A method according to claim 114 in which the compound has the formula (III): 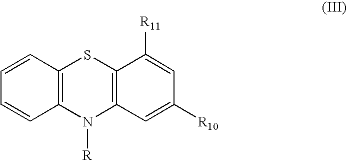
in which R is (CH₂)₃R₁₂; R₁₀ is halogen or C₁-C₄ alkoxy; R₁₁ is hydrogen if R₁₀ is halogen and is hydrogen or methyl if R₁₀ is alkoxy; and R₁₂ is selected from —N(CH₂CH₂)OH, —N(n-C₄H₉), —N(CH₂C₆H₅), 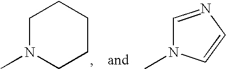
if R₁₀ is halogen, and is —N(CH₃)₂ if R₁₀ is alkoxy.

127. A method according to claim 113 in which the compound is yohimbine.

128. A method according to claim 113 in which the compound is usnic acid.

129. A method according to claim 113 in which the compound is N-{4-[2,5-Dioxo-1-(4-trifluoromethoxy-phenyl)-pyrrolidin-3-yl]-phenyl}-2,2,2-trifluoro-acetamide.

130. A method according to claim 113 in which the compound is a phenothiazine.

131. A method according to claim 113 in which the compound is a thioxanthene.

132. A method according to claim 113 in which the compound is a thianthrene.

133. A method according to claim 113 in which the compound is a phenoxazine.

134. A method according to claim 113 in which the compound is a phenazine.

135. A method according to claim 113 in which the compound is a phenoxathiin.

136. A method according to claim 113 in which the compound is a benzepine.

137. A method for inhibition of microbial RNA function comprising administering to a subject a pharmacologically effective inhibitory amount of
(a) a compound having the formula (I): 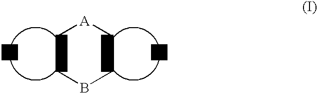
in which:
A is O; NR₁; S(O)ₙ where n is 0, 1 or 2; CR₂R₃; or —A′—CR₄R₅ where A′ is O, NR₁, S(O)ₙ wherein n is 0, 1 or 2 or CR₆R₇, wherein R₁ through R₇ are independently hydrogen or optionally substituted aliphatic or heteroaliphatic groups;
B is N—R, O, S or CR₈R₉ in which R is hydrogen or an optionally substituted aliphatic or heteroaliphatic group and R₈ and R₉ are independently hydrogen or optionally substituted aliphatic or heteroaliphatic groups; or when B is N—R, then R, the nitrogen atom of B, and two carbon atoms on the ring adjacent to the nitrogen atom together comprise a ring having the formula —N—C═C—C(OH)═C(COCH₃)—C(O)—;
provided that at least one of A and B comprises an oxygen, sulfur or nitrogen ring atom;
D and E, together with the carbon atoms to which they are bonded, independently comprise optionally substituted rings of 5-7 atoms selected from C, N, S and O, wherein each ring includes at least one double bond;
and pharmaceutically acceptable salts thereof;
(b) yohimbine;
(c) usnic acid; or
(d) N-{4-[2,5-Dioxo-1-(4-trifluoromethoxy-phenyl)-pyrrolidin-3-yl]-phenyl}-2,2,2-trifluoro-acetamide.

138. A method according to claim 137 in which the compound is of formula (I).

139. A method according to claim 138 in which D and E are unsubstituted rings.

140. A method according to claim 138 in which one of D and E is a mono-substituted ring.

141. A method according to claim 141 in which in which one of D and E is substituted by halogen, trifluoromethyl, cyano, C₁-C₄ alkoxy or acetyl.

142. A method according to claim 138 in which D and E are both mono-substituted rings.

143. A method according to claim 137 in which the compound has the formula (II) 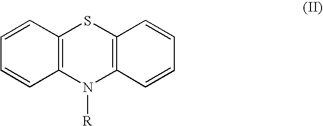
in which
R is hydrogen or an optionally substituted aliphatic, heteroaliphatic or cycloheteroaliphatic group, or in which the nitrogen atom, R, and two carbon atoms on the ring adjacent to the nitrogen atom, together comprise a ring having the formula —N—C═C—C(OH)═C(COCH₃)—C(O)—;
and in which carbon atoms on the side rings of the compound are optionally substituted by one or more of —OR′, ═O, ═S, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NRR′R″)═NR′″, —R′C(NR′R″)═NR′″, —NR—C(NR′R″)═NR′″, —S(O)R′, —S(O)₂R′, —S(O)₂NR′R″, —NRSO₂R′, —CN and/or —NO₂, in which R′, R″ and R′″ are each independently selected from hydrogen, halogen, acyl, optionally substituted heteroaliphatic groups, unsubstituted aryl, aryl substituted with 1-3 halogens, optionally substituted aliphatic, optionally substituted oxyaliphatic groups, optionally substituted thioaliphatic groups, or aryl- (C₁-C₄)aliphatic groups.;
and pharmaceutically acceptable salts thereof.

144. A method according to claim 143 in which both side rings are unsubstituted rings.

145. A method according to claim 143 in which one or both of the side rings is a mono-substituted ring.

146. A method according to claim 145 in which one of the side rings is substituted by halogen, trifluoromethyl, cyano, C₁-C₄ alkoxy or acetyl.

147. A method according to claim 143 in which the side rings are both mono-substituted rings.

148. A method according to claim 143 in which in which the side rings are either unsubstituted or are substituted at the 2-position by halogen, trifluoromethyl, thiomethyl, acetyl, C₁-C₄ alkoxy or cyano, and in which R is
(a) a C₂-C₄ alkylene group substituted by a mono- or dialkylamino group or by an optionally substituted cycloheteroaliphatic group;
(b) an optionally substituted C₂-C₄ unsaturated acyclic aliphatic group, or
(c) a C₂-C₄ acyl group substituted by a mono- or dialkylamino group.

149. A method according to claim 143 in which the compound is acetylpromazine, chlorpromazine, prochlorperazine, promazine, or trifluoperazine.

150. A method according to claim 138 in which the compound has the formula (III): 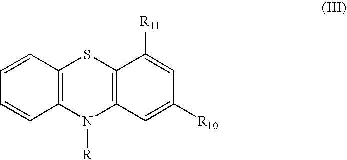
in which R is (CH₂)₃R₁₂; R₁₀ is halogen or C₁-C₄ alkoxy; R₁₁ is hydrogen if R₁₀ is halogen and is hydrogen or methyl if R₁₀ is alkoxy; and R₁₂ is selected from —N(CH₂CH₂)OH, —N(n-C₄H₉), —N(CH₂C₆H₅), 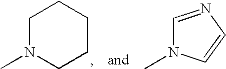
if R₁₀ is halogen, and is —N(CH₃)₂ if R₁₀ is alkoxy.

151. A method according to claim 137 in which the compound is yohimbine.

152. A method according to claim 137 in which the compound is usnic acid.

153. A method according to claim 137 in which the compound is N-{4-[2,5-Dioxo-1-(4-trifluoromethoxy-phenyl)-pyrrolidin-3-yl]-phenyl}-2,2,2-trifluoro-acetamide.

154. A method according to claim 137 in which the compound is a phenothiazine.

155. A method according to claim 137 in which the compound is a thioxanthene.

156. A method according to claim 137 in which the compound is a thianthrene.

157. A method according to claim 137 in which the compound is a phenoxazine.

158. A method according to claim 137 in which the compound is a phenazine.

159. A method according to claim 137 in which the compound is a phenoxathiin.

160. A method according to claim 137 in which the compound is a benzepine.

161. A method according to claim 111 in which the RNA is bacterial RNA.

162. A method for increasing or decreasing the production of a protein comprising the step of contacting a target messenger RNA molecule that encodes said protein with
(a) a compound having the formula (I): 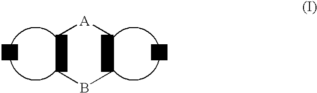
in which:
A is O; NR₁; S(O)ₙ where n is 0, 1 or 2; CR₂R₃; or —A′—CR₄R₅ where A′ is O, NR₁, S(O)ₙ wherein n is 0, 1 or 2 or CR₆R₇, wherein R₁ through R₇ are independently hydrogen or optionally substituted aliphatic or heteroaliphatic groups;
B is N—R, O, S or CR₈R₉ in which R is hydrogen or an optionally substituted aliphatic or heteroaliphatic group and R₈ and R₉ are independently hydrogen or optionally substituted aliphatic or heteroaliphatic groups; or when B is N—R, then R, the nitrogen atom of B, and two carbon atoms on the ring adjacent to the nitrogen atom together comprise a ring having the formula —N—C═C—C(OH)═C(COCH₃)—C(O)—;
provided that at least one of A and B comprises an oxygen, sulfur or nitrogen ring atom;
D and E, together with the carbon atoms to which they are bonded, independently comprise optionally substituted rings of 5-7 atoms selected from C, N, S and O, wherein each ring includes at least one double bond;
and pharmaceutically acceptable salts thereof;
(b) yohimbine;
(c) usnic acid; or
(d) N-{4-[2,5-Dioxo-1-(4-trifluoromethoxy-phenyl)-pyrrolidin-3-yl]-phenyl}-2,2,2-trifluoro-acetamide.

163. A method according to claim 113, wherein increasing or decreasing the production of a protein interferes with the progression of a disease associated with decreasing or increasing the production of said protein, respectively.

164. A method according to claim 114, wherein the disease is selected from the group consisting of amyloidosis, hemophilia, Alzheimer's disease, atherosclerosis, cancer, giantism, dwarfism, hypothyroidism, hyperthyroidism, inflammation, cystic fibrosis, autoimmune disorders, diabetes, aging, obesity, neurodegenerative disorders, and Parkinson's disease.

165. A method according to claim 114, wherein said disease is caused by a bacteria, a fungus, a protozoa, or a virus.

166. A method according to claim 162, wherein the disease is selected from the group consisting of HIV infection, AIDS, human T-cell leukemia, SIV infection, FIV infection, feline leukemia, hepatitis A, hepatitis B, hepatitis C, Dengue fever, malaria, rotavirus infection, severe acute gastroenteritis, diarrhea, encephalitis, hemorrhagic fever, syphilis, legionella, whooping cough, gonorrhea, sepsis, influenza, pneumonia, tinea infection, candida infection, and meningitis.

167. A pharmaceutical composition comprising an amount of
(a) a compound having the formula (I): 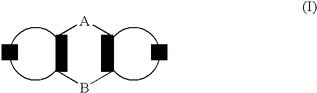
in which:
A is O; NR₁; S(O)ₙ where n is 0, 1 or 2; CR₂R₃; or —A′—CR₄R₅ where A′ is O, NR₁, S(O)ₙ wherein n is 0, 1 or 2 or CR₆R₇, wherein R₁ through R₇ are independently hydrogen or optionally substituted aliphatic or heteroaliphatic groups;
B is N—R, O, S or CR₈R₉ in which R is hydrogen or an optionally substituted aliphatic or heteroaliphatic group and R₈ and R₉ are independently hydrogen or optionally substituted aliphatic or heteroaliphatic groups; or when B is N—R, then R, the nitrogen atom of B, and two carbon atoms on the ring adjacent to the nitrogen atom together comprise a ring having the formula —N—C═C—C(OH)═C(COCH₃)—C(O)—;
provided that at least one of A and B comprises an oxygen, sulfur or nitrogen ring atom;
D and E, together with the carbon atoms to which they are bonded, independently comprise optionally substituted rings of 5-7 atoms selected from C, N, S and O, wherein each ring includes at least one double bond;
and pharmaceutically acceptable salts thereof;
(b) yohimbine;
(c) usnic acid; or
(d) N-{4-[2,5-Dioxo-1-(4-trifluoromethoxy-phenyl)-pyrrolidin-3-yl]-phenyl}-2,2,2-trifluoro-acetamide,
that is pharmacologically effective to inhibit one or more functions of a viral or microbial RNA.

168. A composition according to claim 167 in which the compound is of formula (I).

169. A composition according to claim 168 in which D and E are unsubstituted rings.

170. A composition according to claim 168 in which one of D and E is a mono-substituted ring.

171. A composition according to claim 170 in which in which one of D and E is substituted by halogen, trifluoromethyl, cyano, C₁-C₄ alkoxy or acetyl.

172. A composition according to claim 168 in which D and E are both mono-substituted rings.

173. A composition according to claim 167 in which the compound has the formula (II) 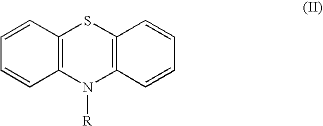
in which
R is hydrogen or an optionally substituted aliphatic, heteroaliphatic or cycloheteroaliphatic group, or in which the nitrogen atom, R, and two carbon atoms on the ring adjacent to the nitrogen atom, together comprise a ring having the formula —N—C═C—C(OH)═C(COCH₃)—C(O)—;
and in which carbon atoms on the side rings of the compound are optionally substituted by one or more of —OR′, ═O, ═S, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NRR′R″)═NR′″, —R′C(NR′R″)═NR′″, —NR—C(NR′R″)═NR′″, —S(O)R′, —S(O)₂R′, —S(O)₂NR′R″, —NRSO₂R′, —CN and/or —NO₂, in which R′, R″ and R′″ are each independently selected from hydrogen, halogen, acyl, optionally substituted heteroaliphatic groups, unsubstituted aryl, aryl substituted with 1-3 halogens, optionally substituted aliphatic, optionally substituted oxyaliphatic groups, optionally substituted thioaliphatic groups, or aryl- (C₁-C₄)aliphatic groups.;
and pharmaceutically acceptable salts thereof.

174. A composition according to claim 173 in which both side rings are unsubstituted rings.

175. A composition according to claim 173 in which one or both of the side rings is a mono-substituted ring.

176. A composition according to claim 175in which in which one of the side rings is substituted by halogen, trifluoromethyl, cyano, C₁-C₄ alkoxy or acetyl.

177. A composition according to claim 173 in which the side rings are both mono-substituted rings.

178. A composition according to claim 173 in which the side rings are either unsubstituted or are substituted at the 2-position by halogen, trifluoromethyl, thiomethyl, acetyl, C₁-C₄ alkoxy or cyano, and in which R is
(a) a C₂-C₄ alkylene group substituted by a mono- or dialkylamino group or by an optionally substituted cycloheteroaliphatic group;
(b) an optionally substituted C₂-C₄ unsaturated acyclic aliphatic group, or
(c) a C₂-C₄ acyl group substituted by a mono- or dialkylamino group.

179. A composition according to claim 173 in which the compound is acetylpromazine, chlorpromazine, prochlorperazine, promazine, or trifluoperazine.

180. A composition according to claim 168 in which the compound has the formula (III): 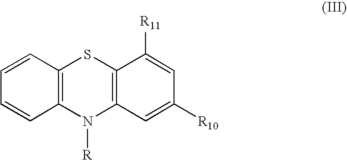
in which R is (CH₂)₃R₁₂; R₁₀ is halogen or C₁-C₄ alkoxy; R₁₁ is hydrogen if R₁₀ is halogen and is hydrogen or methyl if R₁₀ is alkoxy; and R₁₂ is selected from —N(CH₂CH₂)OH, —N(n-C₄H₉), —N(CH₂C₆H₅), 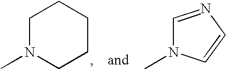
if R₁₀ is halogen, and is —N(CH₃)₂ if R₁₀ is alkoxy.

181. A composition according to claim 167 in which the compound is yohimbine.

182. A composition according to claim 167 in which the compound is usnic acid.

183. A composition according to claim 167 in which the compound is N-{4-[2,5-Dioxo-1-(4-trifluoromethoxy-phenyl)-pyrrolidin-3-yl]-phenyl}-2,2,2-trifluoro-acetamide.

184. A composition according to claim 167 in which the compound is a phenothiazine.

185. A composition according to claim 167 in which the compound is a thioxanthene.

186. A composition according to claim 167 in which the compound is a thianthrene.

187. A composition according to claim 167 in which the compound is a phenoxazine.

188. A composition according to claim 167 in which the compound is a phenazine.

189. A composition according to claim 167 in which the compound is a phenoxathiin.

190. A composition according to claim 167 in which the compound is a benzepine.

191. A pharmaceutical composition comprising an amount of
(a) a compound having the formula (I): 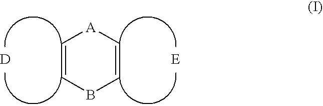
in which:
A is O; NR₁; S(O)ₙ where n is 0, 1 or 2; CR₂R₃; or —A′—CR₄R₅ where A′ is O, NR₁, S(O)ₙ wherein n is 0, 1 or 2 or CR₆R₇, wherein R₁ through R₇ are independently hydrogen or optionally substituted aliphatic or heteroaliphatic groups;
B is N—R, O, S or CR₈R₉ in which R is hydrogen or an optionally substituted aliphatic or heteroaliphatic group and R₈ and R₉ are independently hydrogen or optionally substituted aliphatic or heteroaliphatic groups; or when B is N—R, then R, the nitrogen atom of B, and two carbon atoms on the ring adjacent to the nitrogen atom together comprise a ring having the formula —N—C═C—C(OH)═C(COCH₃)—C(O)—;
provided that at least one of A and B comprises an oxygen, sulfur or nitrogen ring atom;
D and E, together with the carbon atoms to which they are bonded, independently comprise optionally substituted rings of 5-7 atoms selected from C, N, S and O, wherein each ring includes at least one double bond;
and pharmaceutically acceptable salts thereof;
(b) yohimbine;
(c) usnic acid; or
(d) N-{4-[2,5-Dioxo-1-(4-trifluoromethoxy-phenyl)-pyrrolidin-3-yl]-phenyl}-2,2,2-trifluoro-acetamide,
that is pharmacologically effective in inhibiting a viral or microbial infection.

192. A composition according to claim 191 in which the compound is of formula (I).

193. A composition according to claim 193 in which D and E are unsubstituted rings.

194. A composition according to claim 193 in which one of D and E is a mono-substituted ring.

195. A composition according to claim 194 in which in which one of D and E is substituted by halogen, trifluoromethyl, cyano, C₁-C₄ alkoxy or acetyl.

196. A composition according to claim 193 in which D and E are both mono-substituted rings.

197. A composition according to claim 191 in which the compound has the formula (II) 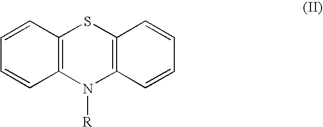
in which
R is hydrogen or an optionally substituted aliphatic, heteroaliphatic or cycloheteroaliphatic group, or in which the nitrogen atom, R, and two carbon atoms on the ring adjacent to the nitrogen atom, together comprise a ring having the formula —N—C═C—C(OH)═C(COCH₃)—C(O)—;
and in which carbon atoms on the side rings of the compound are optionally substituted by one or more of —OR′, ═O, ═S, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NRR′R″)═NR′″, —R′C(NR′R″)═NR′″, —NR—C(NR′R″)═NR′″, —S(O)R′, —S(O)₂R′, —S(O)₂NR′R″, —NRSO₂R′, —CN and/or —NO₂, in which R′, R″ and R′″ are each independently selected from hydrogen, halogen, acyl, optionally substituted heteroaliphatic groups, unsubstituted aryl, aryl substituted with 1-3 halogens, optionally substituted aliphatic, optionally substituted oxyaliphatic groups, optionally substituted thioaliphatic groups, or aryl- (C₁-C₄)aliphatic groups;
and pharmaceutically acceptable salts thereof.

198. A composition according to claim 197 in which both side rings are unsubstituted rings.

199. A composition according to claim 197 in which one or both of the side rings is a mono-substituted ring.

200. A composition according to claim 199 in which in which one of the side rings is substituted by halogen, trifluoromethyl, cyano, C₁-C₄ alkoxy or acetyl.

201. A composition according to claim 197 in which the side rings are both mono-substituted rings.

202. A composition according to claim 197 in which the side rings are either unsubstituted or are substituted at the 2-position by halogen, trifluoromethyl, thiomethyl, acetyl, C₁-C₄ alkoxy or cyano, and in which R is
(a) a C₂-C₄ alkylene group substituted by a mono- or dialkylamino group or by an optionally substituted cycloheteroaliphatic group;
(b) an optionally substituted C₂-C₄ unsaturated acyclic aliphatic group, or
(c) a C₂-C₄ acyl group substituted by a mono- or dialkylamino group.

203. A composition according to claim 197 in which the compound is acetylpromazine, chlorpromazine, prochlorperazine, promazine, or trifluoperazine.

204. A composition according to claim 193 in which the compound has the formula (III): 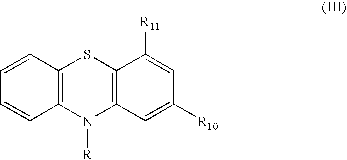
in which R is (CH₂)₃R₁₂; R₁₀ is halogen or C₁-C₄ alkoxy; R₁₁ is hydrogen if R₁₀ is halogen and is hydrogen or methyl if R₁₀ is alkoxy; and R₁₂ is selected from —N(CH₂CH₂)OH, —N(n-C₄H₉), —N(CH₂C₆H₅), 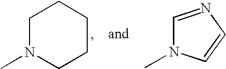
if R₁₀ is halogen, and is —N(CH₃)₂ if R₁₀ is alkoxy.

205. A composition according to claim 191 in which the compound is yohimbine.

206. A composition according to claim 191 in which the compound is usnic acid.

207. A composition according to claim 191 in which the compound is N-{4-[2,5-Dioxo-1-(4-trifluoromethoxy-phenyl)-pyrrolidin-3-yl]-phenyl}-2,2,2-trifluoro-acetamide.

208. A composition according to claim 191 in which the compound is a phenothiazine.

209. A composition according to claim 191 in which the compound is a thioxanthene.

210. A composition according to claim 191 in which the compound is a thianthrene.

211. A composition according to claim 191 in which the compound is a phenoxazine.

212. A composition according to claim 191 in which the compound is a phenazine.

213. A composition according to claim 191 in which the compound is a phenoxathiin.

214. A composition according to claim 191 in which the compound is a benzepine.

215. A compound having the formula (III): 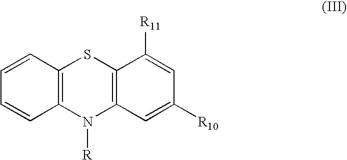
in which R is (CH₂)₃R₁₂; R₁₀ is halogen or C₁-C₄ alkoxy; R₁₁ is hydrogen if R₁₀ is halogen and is hydrogen or methyl if R₁₀ is alkoxy; and R₁₂ is selected from —N(CH₂CH₂)OH, —N(n-C₄H₉), —N(CH₂C₆H₅), 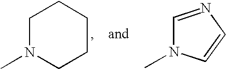
if R₁₀ is halogen, and is —N(CH₃)₂ if R₁₀ is alkoxy.

216. A complex formed between the TAR site of HIV RNA and
(a) a compound having the formula (I): 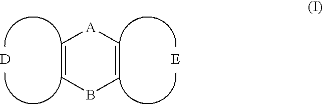
in which:
A is O; NR₁; S(O)ₙ where n is 0, 1 or 2; CR₂R₃; or —A′—CR₄R₅ where A′ is O, NR₁, S(O)ₙ wherein n is 0, 1 or 2 or CR₆R₇, wherein R₁ through R₇ are independently hydrogen or optionally substituted aliphatic or heteroaliphatic groups;
B is N—R, O, S or CR₈R₉ in which R is hydrogen or an optionally substituted aliphatic or heteroaliphatic group and R₈ and R₉ are independently hydrogen or optionally substituted aliphatic or heteroaliphatic groups; or when B is N—R, then R, the nitrogen atom of B, and two carbon atoms on the ring adjacent to the nitrogen atom together comprise a ring having the formula —N—C═C—C(OH)═C(COCH₃)—C(O)—;
provided that at least one of A and B comprises an oxygen, sulfur or nitrogen ring atom;
D and E, together with the carbon atoms to which they are bonded, independently comprise optionally substituted rings of 5-7 atoms selected from C, N, S and O, wherein each ring includes at least one double bond;
and pharmaceutically acceptable salts thereof;
(b) yohimbine;
(c) usnic acid; or
(d) N-{4-[2,5-Dioxo-1-(4-trifluoromethoxy-phenyl)-pyrrolidin-3-yl]-phenyl}-2,2,2-trifluoro-acetamide.
